SEQUENCE LISTING (5 sequences)

SEQ ID NO: 1 - DNA, 29 bp, Artificial
  PCR primer
ggtggtcatatggaagacgccaaaaacat

SEQ ID NO: 2 - DNA, 32 bp, Artificial
  PCR primer
ggtggtgtcgacttacaatttggactttccgc

SEQ ID NO: 3 - DNA, 70 bp, Artificial
  PCR primer
ggtggttctagaaataattttgtttaactttaagaaggagatatacatatgcgtaaaggagaagaacttt

SEQ ID NO: 4 - DNA, 37 bp, Artificial
  PCR primer
ggtggtgtcgacttatttgtatagttcatccatgcca

SEQ ID NO: 5 - RNA, 46 nt, Artificial
  RNA for non-natural peptide synthesis
ggaucgaaagauuuccgcauccccgaaaggguacauggcguuaggu

(19) United States
(12) Patent Application Publication
(10) Pub. No.: US 2012/0171720 A1
(43) Pub. Date: Jul. 5, 2012

(54) Method of Making Ribosomes

(75) Inventors: George M. Church, Brookline, MA (US); Michael C. Jewett, Evanston, IL (US)

(73) Assignee: President and Fellows of Harvard College, Cambridge, MA (US)

(21) Appl. No.: 13/267,470

(22) Filed: Oct. 6, 2011

Related U.S. Application Data
(63) Continuation of application No. PCT/US2010/026379, filed on Mar. 5, 2010.
(60) Provisional application No. 61/166,858, filed on Apr. 6, 2009.

(51) Int. Cl. C12P 21/06; C08H 1/00

(52) U.S. Cl. 435/68.1; 530/409

(57) ABSTRACT

Methods for making in vitro assembled ribosomal subunits and in vitro assembled ribosomes are provided. Methods of transcribing synthetic rRNA and including the synthetic RNA in a synthetic ribosome are provided. Single vessel methods of transcribing synthetic rRNA, forming a synthetic ribosome that includes the synthetic RNA, and allowing the synthetic ribosome to translate a protein are also provided. Methods of screening for novel, synthetic ribosomal subunits and/or ribosomes are provided. Synthetic replicons and methods of making synthetic replicons are also provided.

### RELATED APPLICATIONS

This application is a continuation of PCT application number PCT/US2010/026379 designating the United States and filed Mar. 5, 2010; which claims the benefit of U.S. provisional patent application No. 61/166,858, filed Apr. 6, 2009, which is hereby incorporated herein by reference in its entirety for all purposes.

### STATEMENT OF GOVERNMENT INTERESTS

This invention was made with government support under EEC-0540879 awarded by the National Science Foundation and GM081450 awarded by the National Institutes of Health. The government has certain rights in the invention.

### FIELD

Embodiments of the present invention relate in general to methods of making ribosomes in physiologically compatible media from component parts. Embodiments of the present invention further include methods of making natural and non-natural biopolymers using a ribosome made from component parts in physiologically compatible media.

### BACKGROUND

The ribosome is one of the largest macromolecular and functionally important complexes of the cell. Previous works have shown total reconstitution of functionally active 30S and 50S subunits from native E. coli rRNA and proteins (Traub P, Nomura M (1968) Structure and function of E. coli ribosomes. V. Reconstitution of functionally active 30S ribosomal particles from RNA and proteins. Proc. Natl. Acad. Sci. USA 59: 777-784; Nierhaus K H, Dohme F (1974) Total reconstitution of functionally active 50S ribosomal subunits from Escherichia coli. Proc. Natl. Acad. Sci. USA 71: 4713-4717). However, reconstituted ribosomes have only ever been shown competent for their ability to translate poly-uridine [Poly(U)] mRNA templates into polyphenylalanine. Moreover, reconstituting E. coli ribosomes under physiological conditions has remained elusive for decades. 50S ribosome reconstitutions performed using methods known in the art are non-physiological. E. coli ribosomes that have been reconstituted with in vitro transcribed 23S rRNA have been shown to be less active than the natural version in N-Ac-Met-puromycin synthesis, and reconstitutions have been shown to be less efficient. Further, previous protocols have not attempted to make a ribosome in situ.

Construction of E. coli ribosomes requires the synthesis and assembly of 21 small subunit ribosomal(r)-proteins, 33 large subunit r-proteins, and 3 rRNAs (23S, 16S, and 5S rRNA) (FIG. 21). In vitro assembly of these components into functionally active small (30S) and large (50S) ribosomal subunits was first achieved about 40 years ago (Nierhaus, K. H. & Dohme, F., Total reconstitution of functionally active 50S ribosomal subunits from Escherichia coli. Proc Natl Acad Sci USA 71, 4713-4717 (1974); Traub, P. & Nomura, M., Structure and function of E. coli ribosomes. V. Reconstitution of functionally active 30S ribosomal particles from RNA and proteins. Proc Natl Acad Sci USA 59, 777-784 (1968)). The 30S subunit reconstitution protocol involves a one-step incubation at 40° C. (FIG. 22A), and can be facilitated at lower temperatures by chaperones (Maki, J. A. & Culver, G. M., Recent developments in factor-facilitated ribosome assembly. Methods 36, 313-320 (2005)). The classical 50S subunit reconstitution protocol involves a two-step high temperature incubation, first at 4 mM Mg²⁺ and 44° C., then at 20 mM Mg²⁺ and 50° C. (FIG. 22B). Unfortunately, these reconstitution methods are not compatible with protein synthesis, requiring separate reactions for ribosome self-assembly and functional activity (i.e., peptide bond formation). In addition, even though in vitro synthesized rRNA has been substituted for the in vivo derived version (Green, R. & Noller, H. F., In vitro complementation analysis localizes 23S rRNA posttranscriptional modifications that are required for Escherichia coli 50S ribosomal subunit assembly and function. Rna 2, 1011-1021 (1996); Semrad, K. & Green, R., Osmolytes stimulate the reconstitution of functional 50S ribosomes from in vitro transcripts of Escherichia coli 23S rRNA. Rna 8, 401-411 (2002); Cunningham, P. R., Richard, R. B., Weitzmann, C. J., Nurse, K., & Ofengand, J., The absence of modified nucleotides affects both in vitro assembly and in vitro function of the 30S ribosomal subunit of Escherichia coli. Biochimie 73, 789-796 (1991)), combined rRNA synthesis and ribosome self-assembly has not been achieved as it occurs in vivo (Nierhaus, K. H., The assembly of prokaryotic ribosomes. Biochimie 73, 739-755 (1991); Talkington, M. W., Siuzdak, G., & Williamson, J. R., An assembly landscape for the 30S ribosomal subunit. Nature 438, 628-632 (2005)).

### SUMMARY

In vitro systems offer a powerful approach to understand complex biological processes and to expand natural capabilities for the production of native and synthetic biomolecules (Forster, A. C. & Church, G. M., Synthetic biology projects in vitro. Genome Res 17, 1-6 (2007); Davidson, E. A., Dlugosz, P. J., Levy, M., & Ellington, A. D., Directed evolution of proteins in vitro using compartmentalization in emulsions. Curr Protoc Mol Biol Chapter 24, Unit 24 26 (2009); Swartz, J., Developing cell-free biology for industrial applications. J Ind Microbiol Biotechnol 33, 476-485 (2006); Wang, L., Xie, J., & Schultz, P. G., Expanding the genetic code. Annu Rev Biophys Biomol Struct 35, 225-249 (2006)). These systems are not constrained by cellular complexity, structural barriers, and viability, yet they lack the most salient feature of biology, self-replication. Cell-free replication could enable evolutionary optimization to address challenges beyond those found in nature for a variety of therapeutic, diagnostic, and research applications. Without intending to be bound by scientific theory, based on the data discussed further herein, it is possible that approximately 151 biomolecular components from Escherichia coli and its bacteriophages may be sufficient to enable rapid and accurate self-replication in vitro (Forster, A. C. & Church, G. M., Towards synthesis of a minimal cell. Mol Syst Biol 2, 45 (2006)). However, efforts to construct such a system have been precluded by the inability to synthesize and assemble E. coli ribosomes under conditions that are compatible with in vitro transcription and translation.

Novel methods for physiological construction of ribosomes are described herein. In contrast to existing two-step methods that have remained fairly constant for decades (Nierhaus, K. H., Reconstitution of ribosomes, in Ribosomes and Protein Synthesis, A Practical Approach. (Oxford University Press, Oxford, 1990)), in accordance with aspects of the present invention, E. coli ribosomes are reconstituted in a one-step incubation procedure at 37° C. under chemical conditions that mimic the cytoplasm. According to certain aspects, ribosome self-assembly in physiological conditions is combined with transcription and translation in a single, cell-free reaction. Ribosomal RNA (rRNA) synthesis is also combined with ribosome self-assembly to make functionally active ribosomes. The integrated approach described herein solves critical barriers to constructing and evolving novel ribosomes in vitro. The methods and compositions described herein enable cell-free replication as a complement to in vivo based efforts for engineering whole genomes and programming synthetic organisms (Gibson, D. G. et al., Complete chemical synthesis, assembly, and cloning of a Mycoplasma genitalium genome. Science 319, 1215-1220 (2008); Wang, H. H. et al., Programming cells by multiplex genome engineering and accelerated evolution. Nature (2009)) and as a route towards synthetic life (Forster, A. C. & Church, G. M., Towards synthesis of a minimal cell. Mol Syst Biol 2, 45 (2006); Luisi, P. L., Ferri, F., & Stano, P., Approaches to semi-synthetic minimal cells: a review. Naturwissenschaften 93, 1-13 (2006); Szostak, J. W., Bartel, D. P., & Luisi, P. L., Synthesizing life. Nature 409, 387-390 (2001)).

The ribosome is the most conserved and complex molecular machine in living systems. It is a key component to the biotechnology industry in that it is necessary to make proteins which are in turn necessary to make metabolites, RNAs, materials and the like. Cell-free protein synthesis is already a viable industry useful for making purer proteins, omitting degradative enzymes, changing the genetic code, making cell-toxic proteins or making proteins under conditions incompatible with cells. The methods described herein manipulate the ribosome, enabling selection for new and/or improved protocols exploiting design and/or evolution of ribosomes.

Embodiments of the present invention are directed to methods of making a functional ribosome by combining together component parts. According to certain aspects of the present invention, the component parts of a ribosome can be tailored to achieve a particular ribosome design when assembled according to the methods of the present invention. The component parts of the ribosome can be naturally occurring, or synthetic versions of naturally occurring components or synthetic non-natural components. The ribosome can be designed to manufacture any particular biopolymer based on the selection and/or design of the component parts. For example, using the methods of the present invention, a ribosome can be designed to produce a naturally occurring biopolymer such as a nucleic acid or a polypeptide using naturally occurring, or synthetic versions of naturally occurring components or synthetic non-natural components. Additionally, using the methods of the present invention, a ribosome can be designed to produce a non-naturally occurring biopolymer or other polymer based on nucleotides or amino acids whether naturally occurring or synthetic derivatives using naturally occurring, or synthetic versions of naturally occurring components or synthetic non-natural components. According to the present invention, methods are presented to tailor-make ribosomes that produced desired polymers. According to this aspect of the present invention, synthetic ribosomes are designed as factories for the manufacture of desired polymers.

In certain exemplary embodiments, a method of making an in vitro assembled ribosomal subunit is provided. The method includes the steps of providing polypeptides that assemble to form a ribosomal subunit, contacting the polypeptides that assemble to form the ribosomal subunit with ribosomal RNAs (rRNAs), incubating the polypeptides and rRNAs at a constant temperature and a constant Mg²⁺ concentration, and allowing assembly of the ribosomal subunit. In certain aspects, the incubating is performed at about 37° C. In other aspects, the Mg²⁺ concentration is about 20 mM. In other aspects, the ribosomal subunit is a 50S subunit or a 30S subunit. In yet other aspects, the rRNAs are natural or synthetic or a combination thereof. In still other aspects, the polypeptides are natural, synthetic or a combination thereof.

In certain exemplary embodiments, a method of making an in vitro assembled ribosome is provided. The method includes the steps of providing polypeptides that assemble to form one or more ribosomal subunits, transcribing synthetic rRNA in the presence of the polypeptides that assemble to form the ribosomal subunits, and allowing the ribosome to self-assemble. In certain aspects, the synthetic rRNA is selected from the group consisting of 16S rRNA, 23S rRNA, 5S rRNA or any combination thereof. In other aspects, the polypeptides and rRNA assemble to form one or both of the 30S subunit and the 50S subunit.

In certain exemplary embodiments, a method of in vitro translation is provided. The method includes the steps of providing polypeptides that assemble to form ribosomal subunits in a vessel, providing transcription reagents and a nucleic acid sequence that encodes rRNA in the vessel, providing tRNA, a polymerase, NTPs, amino acids and a nucleic acid sequence encoding a protein in the vessel, allowing assembly of ribosomal subunits and rRNA to form a ribosome, and allowing the ribosome to translate the protein encoded by the nucleic acid sequence. In certain aspects, the contents of the vessel are incubated at a temperature between about 30° C. and about 37° C., or at about 37° C. In other aspects, the contents of the vessel are incubated in the presence of Mg²⁺ present at a concentration between about 10 mM and about 25 mM, or at about 14 mM. In certain aspects, at least some natural rRNAs are provided in the vessel. In other aspects, all the rRNAs are transcribed in the vessel. In other aspects, a sequence-defined non-natural or natural biopolymer is synthesized. In yet other aspects, the protein has one or more biological activities such as, e.g., an enzymatic activity, a pharmaceutical activity and the like.

In certain exemplary embodiments, a method of making a pharmaceutical compound is provided. The method includes the steps of translating ribosomal proteins that assemble to form one or more ribosomal subunits, transcribing synthetic rRNA in the presence of the ribosomal proteins to form the ribosomal subunits, allowing the ribosome to self-assemble, providing a nucleic acid sequence encoding a pharmaceutical compound, and allowing the ribosome to translate the nucleic acid sequence to produce the pharmaceutical compound. In certain aspects, the pharmaceutical compound is an non-natural biopolymer, is a natural biopolymer or is a combination thereof. In other aspects, the pharmaceutical compound contains one or more D-amino acids.

In certain exemplary embodiments, a method of making an in vitro assembled ribosome is provided. The method includes the steps of providing ribosomal proteins that assemble to form one or more ribosomal subunits, transcribing synthetic rRNA in the presence of the ribosomal proteins to form the ribosomal subunits, and allowing the ribosome to self-assemble. In certain aspects, the synthetic rRNA is selected from the group consisting of 16S rRNA, 23S rRNA, 5S rRNA or any combination thereof. In other aspects, the ribosomal proteins include any one or more of known ribosomal proteins or all known ribosomal proteins. In yet other aspects, the ribosomal proteins and the rRNA assemble to form one or both of 30S subunit and 50S subunit. In certain aspects the ribosomal proteins are synthetic.

In certain exemplary embodiments, a method of making an in vitro assembled ribosome is provided. The method includes the steps of translating the ribosomal proteins in the presence of rRNA that assemble to form one or more ribosomal subunits, providing rRNA that assembles to form the ribosomal subunits, and allowing the ribosome to self-assemble. In certain aspects, the synthetic rRNA is selected from the group consisting of 16S rRNA, 23S rRNA, 5S rRNA or any combination thereof. In other aspects, the ribosomal proteins include any one or more of known ribosomal proteins or all known ribosomal proteins. In yet other aspects, the ribosomal proteins and the rRNA assemble to form one or both of 30S subunit and 50S subunit.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 graphically depicts the activities of reconstituted 50S and 30S ribosomal subunits using a one-step reconstitution method. Reagent concentrations listed in parentheses were as follows: (T (° C.), Mg²⁺ (mM), NH₄⁺ (mM), K⁺ (mM), Cl⁻ (mM), OAc⁻ (mM), Glu⁻ (mM), spermidine (mM), putrescine (mM), chaperones (DnaJ, DnaK, GrpE, GroEL, GroES)). “R” denotes reconstituted.

FIG. 2 graphically depicts the activities of reconstituted 50S and 30S ribosomal subunits that have been reconstituted at constant temperature and magnesium concentrations. Reagent concentrations listed in parentheses were as follows: (T (° C.), Mg²⁺ (mM), NH₄⁺ (mM), K⁺ (mM), Cl⁻ (mM), OAc⁻ (mM), Glu⁻ (mM), spermidine (mM), putrescine (mM), chaperones (DnaJ, DnaK, GrpE, GroEL, GroES). “R” denotes reconstituted.

FIG. 3 schematically depicts rRNA in vitro synthesis and assembly.

FIG. 4 schematically and graphically depicts that in vitro transcribed 16S rRNA coupled with self-assembly generates functional ribosomes. Conditions were as follows: 37° C., 3 hours, 14 mM Mg²⁺, n=6.

FIG. 5 schematically and graphically depicts that in vitro transcribed 23S rRNA coupled with self-assembly generates functional ribosomes. Conditions were as follows: 37° C., 3 hours, 14 mM Mg²⁺, n=6.

FIG. 6 schematically and graphically depicts that in vitro transcribed 23 S rRNA and 16S rRNA coupled with self-assembly generates functional ribosomes. Conditions were as follows: 37° C., 3 hours, 14 mM Mg²⁺, n=6.

FIG. 7 schematically depicts the components for creating a synthetic, self-replicating ribosome.

FIG. 8 sets forth experimental conditions for using E. coli cell extracts and/or pure or partially pure proteins for modifying rRNA and/or facilitating rRNA folding and/or assembly.

FIGS. 9A-9B depict parameters for constructing modified ribosomes for incorporation of D-amino acids into proteins (A) (Hecht (2006) Biochemistry) and a highly flexible tRNA acylation method for non-natural peptide synthesis (B) (Suga (2006) Nat. Methods).

FIGS. 10A-10B graphically depict that ribosomes from synthetic 23S rRNA with coupled translation can generate active luciferase. “R” denotes reconstituted.

FIGS. 11A-11B graphically depict that reconstituted 30S ribosomal subunits coupled with native 50S subunits express active luciferase (A), and that reconstituted 50S ribosomal subunits coupled with native 30S subunits express active luciferase (B). “R” denotes reconstituted; 3 hour batch reaction.

FIGS. 12A-12B graphically depict activities of reconstituted ribosomal subunits. (A) 70S ribosomal subunits can be reconstituted at 37° C. under conditions that mimic the cytoplasm. (B) Reconstituted 70S ribosomes express active luciferase, two hour batch reaction. “R” denotes reconstituted.

FIGS. 13A-13C graphically depict whether ribosomal subunits can be reconstituted and synthesize protein in the same compartment. (A) 30S ribosomal subunits can be reconstituted and synthesize protein in the same compartment. (B) 50S ribosomal subunits can be reconstituted and synthesize protein in the same compartment. (C) 70S ribosomal subunits can be reconstituted and synthesize protein in the same compartment. (A)-(C) performed at 37° C. for 3 hours in the presence of 14 mM Mg²⁺, n=4.

FIG. 14 schematically depicts a strategy for isolating active synthetic 23S rRNA including the following steps: a) design and synthesize 23S rRNA genes; b) in vitro transcription and pool purification; c) 50S reconstitution; d) 70S formation and purified translation with mRNA-protein-ribosome complex formation; e) selection by affinity purification; f) purify, sequence functional analysis (e.g., polysomes); g) re-design and/or mutagenize; optionally repeat steps b)-g) one or more times.

FIGS. 15A-15B graphically depict the effects of various experimental conditions on the ability of reconstituted ribosomal subunits on translation.

FIG. 16 graphically depicts the effects of various experimental conditions on the ability of reconstituted 50S ribosomal subunits coupled with native 30S subunits to express active green fluorescent protein (GFP). Reagent concentrations listed in parentheses were as follows: (T (° C.), Mg²⁺ (mM), NH₄⁺ (mM), K⁺ (mM), Cl⁻ (mM), OAc⁻ (mM), Glu⁻ (mM), spermidine (mM), putrescine (mM), chaperones (DnaJ, DnaK, GrpE, GroEL, GroES)). “R” denotes reconstituted.

FIG. 17 graphically depicts protein synthesis kinetics for reconstituted 50S subunits. “R” denotes reconstituted.

FIG. 18 graphically depicts data showing that in vitro transcribed 23S rRNA coupled with self-assembly generates functional ribosomes.

FIG. 19 schematically depicts a method for screening libraries (e.g., rRNA libraries).

FIG. 20 schematically depicts a method for screening for novel ribosomes and/or ribosomal subunits.

FIG. 21 schematically depicts synthesis and self-assembly of functional ribosomes as a step towards cell-free replication. A strategy for self-replication of a mini-genome (which is expected to be larger than a fully “minimal” genome) dependent only on small molecule substrates is shown. The cell-free system described herein reconstitutes the macromolecular catalysts synthesizing DNA, RNA, and protein and demands in vitro methods for production and self-assembly of active component parts. Focus is given towards ribosome synthesis and self-assembly (solid arrows). DNA pol: DNA polymerase; RNA Pol: RNA polymerase; TFs: translation factors; aaRSs: aminoacyl-tRNA synthetases.

FIGS. 22A-22E depicts data showing that ribosomes reconstituted using classical methods synthesize active firefly luciferase (Flue). (A) Schematic of traditional ribosome reconstitution methods. (B) Bioluminescence (in relative light units (RLUs)) and luciferase molecules (fmoles) synthesized (4000 RLU is approximately 1 fmol) from cell-free protein synthesis reactions where 30S reconstitution mixtures (R30S) were paired with native 50S subunits. (C) Calculated from (B), the moles of active Fluc peptide bonds synthesized per moles of maximum ribosome equivalents (m.r.e.). Because the reaction contained 1.5 pmol of R305 subunits and 3 pmol of 50S subunits, the m.r.e.=1.5 pmol (at most 1.5 pmol 70S ribosomes can be translating). (D) Fluc peptide bond synthesis from 50S reconstitution mixtures (R50S) (m.r.e.=0.75 pmol). (E) Fluc peptide bond synthesis from R50S and R30S (m.r.e.=0.75 pmol). Here, subunit reconstitutions were carried out separately and then each reconstitution mixture was added to the cell-free protein synthesis reaction. Data represent the average of 15≧n≧6 independent experiments. Error bars=1 s.d.

FIG. 23 depicts that E. coli ribosomes reconstituted under physiological conditions synthesize active Fluc. The dependence of protein synthesis activities of 50S reconstitution mixtures is shown for three factors: (1) the concentration of the major ionic component, (2) temperature, and (3) magnesium concentration. Filled and open rectangles indicate the presence or absence, respectively, of the specified factor in the reconstitution reaction. For the two-step procedure (e.g., 44° C.→50° C.), 20 minute incubation was followed by 90 minute incubation. For the one-step procedure, reconstitution was carried out for 120 minutes. Fluc peptide bond synthesis from 50S reconstitution mixtures is shown. Negative control reactions (e.g. TP50 paired with 30S subunits) gave immeasurable results over all conditions. Data represent the average of 15≧n≧4 independent experiments. Error bars=1 s.d.

FIGS. 24A-24D depict that ribosome self-assembly and protein synthesis are integrated to produce active Fluc. (A) In contrast to two separate reactions ((1) reconstitution and (2) combined transcription and translation), ribosome self-assembly and protein synthesis were carried out together for 180 minutes at 14 mM Mg²⁺ and 37° C. (B) 30S subunits self-assembled and paired with native 50S subunits to synthesize Fluc in one reaction. (C) 50S subunits self-assembled and paired with native 30S subunits to synthesize Fluc in one reaction. (D) 70S ribosomes self-assembled and synthesized Fluc in a single reaction. Data represent the average of 12≧n≧6 independent experiments. Results are plotted on a log-scale. Error bars=1 s.d.

FIGS. 25A-25D depict the physiological construction of functionally active ribosomes. (A) integrated rRNA synthesis, ribosome self-assembly, and protein synthesis reactions were carried out for 180 minutes at 14 mM Mg²⁺ and 37° C. (B) 50S subunits self-assembled into active ribosomes from in vitro transcribed 23S rRNA and native TP30, 5S rRNA and 30S subunits. (C) 30S subunits self-assembled into active ribosomes from in vitro transcribed 16S rRNA and native TP30 and 50S subunits. (D) Functionally active 70S ribosomes self-assembled from in vitro transcribed 23S rRNA and 16S rRNA and native 5S rRNA, TP50 and TP30. Data represent the average of 16≧n≧12 independent experiments. Results are plotted on a log-scale. Error bars=1 s.d.

FIGS. 26A-26B graphically depict data that show that 50S ribosomal subunits reconstituted using classical methods synthesize green fluorescent protein (GFP). (A) GFP production in 150 μL combined transcription and translation reactions where 0.75 pmol 50S reconstitution mixtures (R50S) were paired with 3 pmol native 30S subunits (m.r.e.=0.75 pmol). Fluorescence was measured following 2 hour incubation at 30° C. and 8 hour maturation at 4° C. Fluorescence was converted to an estimate of protein concentration using a standard curve created from dilutions of purified GFP (4000 Fluorescence units (arbitrary) equal approximately 1 ng GFP). R50S subunits showed approximately 20% of the native subunit activity. Control transcription and translation reactions without ribosomes or without GFP template DNA had immeasurable fluorescence. Protein synthesis was immeasurable when only 23S and 5S rRNA or TP50 was used for reconstitution. (B) Calculated from (A), the moles of active GFP peptide bonds synthesized per moles of m.r.e. Reconstituted ribosomes synthesized about the same moles of peptide bonds per m.r.e. of Fluc and GFP (see FIG. 22D). Since the Fluc assay was more sensitive, Fluc was chosen as a model protein for the experiments described herein. Data represent the average of n=4 independent experiments. Error bars=1 s.d.

FIG. 27 graphically depicts data showing protein synthesis kinetics with classical 50S subunit reconstitution mixtures. Following classical reconstitution, protein synthesis reactions were carried out for 2 hours at 30° C. Fifteen microliter reactions were prepared in different tubes for each time point. Data represent the average of n=4 independent experiments. Error bars=1 s.d.

FIGS. 28A-28B depict the dependence of protein synthesis activity of 50S reconstitution mixtures on the ionic environment. (A) Classical two-step 50S reconstitutions with ammonium acetate substituted for ammonium chloride. (B) Classical two-step 50S reconstitutions with potassium glutamate substituted for ammonium chloride. Following reconstitution, protein synthesis reactions were carried out for 2 hours at 30° C. Substituting ammonium acetate for ammonium chloride reduced reconstitution efficiency. Substituting potassium glutamate for ammonium chloride did not impact reconstitution efficiency at optimum values. Data represent the average of n=4 (A) or n=6 (B) independent experiments. Error bars=1 s.d.

FIG. 29 depicts that Fluc production from reconstituted ribosomes was markedly decreased when the chemical composition of the classical poly(Phe) translation system was substituted for the cell-free protein synthesis system described herein. Although focusing on reconstitution and ribosome self-assembly protocols, major differences also exist between the translation system described herein and that used in previous works (Table 1). In this figure, protein synthesis activity of reconstituted 50S subunit mixtures derived from different protocols were tested in the chemical composition of the classical poly(Phe) translation system reported by Nierhaus (Supra). Each reconstitution included a two-step [Mg2+] change from 4 mM 20 mM. Filled and open rectangles indicate the presence or absence, respectively, of the specified factor in the reconstitution reaction. Fluc synthesis using ribosomes reconstituted with 400 mM ammonium chloride was immeasurable. In contrast, protein synthesis activity with 50S ribosomes reconstituted with glutamate salts synthesized active protein, suggesting advantages for the methods described herein. Of note, the combined transcription and translation system designed to mimic the cytoplasm that is described herein was approximately 200-fold more productive in the two-step temperature protocol and approximately 800-fold more productive in the isothermal protocol than when the ionic environment of the classic poly(Phe) system was used (See FIG. 23 to compare). These results demonstrate the importance of developing a coordinated ribosome assembly and protein synthesis platform. Data represent the average of n=4 independent experiments. Error bars=1 s.d.

FIG. 30 depicts data showing that 50S subunits reconstituted under physiological conditions synthesize active Fluc. The dependence of protein synthesis activities of 50S reconstitution mixtures on the concentration of the major ionic component, temperature, chaperones and polyamines (1.5 mM spermidine and 1 mM putrescine) is shown. Each reconstitution included a two-step [Mg2+] change from 4 mM to 20 mM. After reconstitution, protein synthesis reactions were carried out for 2 hours at 30° C. When testing the impact of chaperone addition, 1.5 pmol DnaK, 1.5 pmol DnaJ, 3 pmol GrpE, 1.5 pmol GroEL, and 1.5 pmol GroES were added for 0.75 pmol 23S rRNA, along with 1 mM ATP. Filled and open rectangles indicate the presence or absence, respectively, of the specified factor in the reconstitution reaction. Isothermal reconstitution was less efficient. Use of chaperones or polyamines did not stimulate 50S reconstitution. Follow-on experiments showed that these factors also did not stimulate 70S reconstitution (not shown). It should be noted that the S150 extract (which presumably contained active chaperones (Davidson, Supra)) and/or the physicochemical environment of the protein synthesis system (which included 1.5 mM spermidine and 1 mM putrescine) could potentially affect the results. Data represent the average of n=4 independent experiments. Error bars=1 s.d.

FIG. 31 depicts that E. coli ribosomes reconstituted under physiological conditions synthesized active Fluc from 70S reconstitution mixtures. Filled and open rectangles indicate the presence or absence, respectively, of the specified factor in the reconstitution reaction. After reconstitution, protein synthesis reactions were carried out for 2 hours at 30° C. Data represent the average of 15≧n≧4 independent experiments. Error bars=1 s.d.

FIGS. 32A-32F depict the impact of temperature and magnesium concentration on combined ribosome self-assembly and protein synthesis. (A) schematic of combined ribosome self-assembly and protein synthesis. Temperature and magnesium concentrations were varied to identify parameters leading to the highest level of peptide bond polymerization per m.r.e. Temperatures shown include 30° C. (the optimum temperature for active Fluc synthesis) and 37° C. (the physiological temperature for optimum E. coli growth and the temperature used in one-step reconstitution experiments). Magnesium concentrations shown include 20 mM Mg²⁺ (the experimentally determined optimum for classical 30S and 50S reconstitution) and 14 mM Mg²⁺ (the experimentally determined optimum for the cell-free transcription and translation system). The experimentally determined magnesium concentration optimum was higher than those typically used for in vitro translation systems composed of purified components (approximately 5 mM) (Jelenc, Supra; Pavlov, Supra), although somewhat similar to the poly(Phe) system of Nierhaus (Supra) (Table 1) and to the PURE translation system (Shimizu (2001), Supra; Shimizu (2005), Supra). Without intending to be bound by scientific theory, this discrepancy likely resulted from the use of a cell-free protein synthesis system designed to mimic the intracellular physicochemical environment (Jewett (2008), Supra; Jewett (2004), Supra). While glutamate (the most predominant anion of the cell) is not generally used in purified translation systems, the concentration of glutamate in the cell-free system used described herein is approximately 150 mM. Given that the reported log stability constant for [MgGlu-]/[Mg][Glu] is 1.99, it seems reasonable that 150 mM glutamate would have a strong buffering effect on the concentration of free Mg²⁺ and would therefore impact the observed optimum for total [Mg²⁺]. In these experiments, all positive control reactions used 0.75 pmol native 50S subunits and 3 pmol native 30S subunits. As a result, the yield of Fluc appeared higher for native 30S subunits relative to reconstituted 30S subunits in (B) and (C) than previously observed (FIG. 22) because the molar ratio of 50S:30S subunits is 1:4 rather than 2:1. (B), (C) the dependence of 30S subunit self-assembly and protein synthesis on temperature and magnesium concentration. The moles of active Fluc peptide bonds synthesized per mole of m.r.e. when 1.5 pmol R30S subunits were paired with 3 pmol native 50S subunits. (D), (E) the dependence of 50S subunit self-assembly and protein synthesis on temperature and magnesium concentration. The moles of active Fluc peptide bonds synthesized per mole of m.r.e. when 0.75 pmol R50S subunits were paired with 3 pmol native 30S subunits. (F) the dependence of 70S ribosome self-assembly and protein synthesis system on temperature (m.r.e.=0.75 pmol). Protein synthesis from self-assembly of 70S ribosomes at 20 mM Mg²⁺ was not measurable. In all cases, combined reconstitution and protein synthesis activity was higher at 37° C. and 14 mM Mg²⁺. Data represent the average of n=4 independent experiments. Error bars=1 s.d.

FIGS. 33A-33B depict data that demonstrate that the synthesis of non-specific rRNA in reactions integrating rRNA synthesis, ribosome self-assembly and protein synthesis indicates that possible contaminating native rRNA is not masked by excess ribosomal proteins. To test that the activity of the in vitro transcribed rRNA was not the result of unmasking some minor level of contaminating native rRNA from the purified TP50 or TP30 mixtures (as previously observed in the RNAse P field (Gold, H. A. & Altman, S., Reconstitution of RNAase P activity using inactive subunits from E. coli and HeLa cells. Cell 44, 243-249 (1986)), rRNA swapping experiments were carried out as inbuilt controls. Since unmasking may conceivably have some sequence specificity, it should be noted that there was an excess of tRNA and luciferase mRNA in the reactions. Moreover, unmasking has not previously been observed when using in vitro transcribed rRNA to assemble ribosomes (Green, R. & Noller, H. F., In vitro complementation analysis localizes 23S rRNA posttranscriptional modifications that are required for Escherichia coli 50S ribosomal subunit assembly and function. Rna 2, 1011-1021 (1996); Semrad, K. & Green, R., Osmolytes stimulate the reconstitution of functional 50S ribosomes from in vitro transcripts of Escherichia coli 23S rRNA. Rna 8, 401-411 (2002)). (A) 23S rRNA was transcribed in 30S subunit synthesis and self-assembly reactions by substituting plasmid pCW1 (encoding the gene for 23S rRNA) for pWK1 (encoding the gene for 16S rRNA). (B) 16S rRNA was transcribed in 50S subunit synthesis and self-assembly reactions by substituting plasmid pWK1 for pCW1. Doping in purified, naturally derived 16S rRNA into 50S assembly reactions with in vitro transcribed 23S rRNA had no effect. Data represent the average of n=4 independent experiments. Error bars=1 s.d.

FIG. 34 graphically depicts that luciferase production from an independent set of 70S ribosomes demonstrated high ribosome quality. Since the moles of luciferase produced per m.r.e. in the cell-free protein synthesis system indicated that only approximately 5-15% of native ribosomes translated active full-length protein (which is consistent with historical observations from crude extract translation systems), ribosome quality was tested against an independent set of highly active coupled 70S ribosomes that had been purified according to Rodnina et al. (Rodnina, M. V. & Wintermeyer, W., GTP consumption of elongation factor Tu during translation of heteropolymeric mRNAs. Proc Natl Acad Sci USA 92, 1945-1949 (1995)). Combined transcription and translation reactions with 0.5 μM 70S ribosomes were carried out at 37° C. for 3 hours. Synthesis of active luciferase was determined by enzymatic assay and ribosomes used herein were more active, indicating that the low ribosome utilization observed was not due to poor ribosome quality. Data represent the average of n=4 independent experiments. Error bars=1 s.d.

FIG. 35 schematically depicts a synthetic replicon.

FIGS. 36A-36C graphically depict ribosome synthesis and self-assembly using a PURE translation system lacking ribosomes instead of an S150 extract (Shimizu et al. (2001) Nat. Biotechnol. 19:751; the PURE system is commercially available from New England Biolabs, Beverly, Mass.). (A) 50S, (B) 30S, (C) 70S.

FIGS. 37A-37C graphically depict rRNA synthesis, ribosome self-assembly and protein synthesis comparing the PURE translation system lacking ribosomes and the S150 extracts. (A) 50S, (B) 70S, (C) 30S.

### DETAILED DESCRIPTION

The principles of the present invention are based on methods to reconstitute ribosomes, whether prokaryotic or eukaryotic. In certain aspects, the reconstituted ribosomes described herein can synthesize active, full-length protein, e.g., luciferase and/or green fluorescent protein. Furthermore, active E. coli ribosomes can be reconstituted in a one-step incubation procedure at 37° C. under conditions that mimic the cytoplasm. Finally, in vitro transcribed 16S rRNA and 23S rRNA, combined with native ribosomal proteins and native 5S rRNA, self-assemble into functional synthetic ribosomes.

According to certain exemplary embodiments, a synthetic replicon is provided. In certain exemplary embodiments methods and compositions for rRNA synthesis, ribosome assembly and protein synthesis in one vessel are provided. The reconstitution methods described herein facilitate the in vitro analysis of ribosomal mutations for understanding the molecular details of ribosome function. The reconstitution methods described herein solve one of the critical barriers to constructing ribosomes in vitro, and enable cell-free synthetic biology as a platform for evolving ribosomes for the production of protein therapeutics and peptide drugs that are difficult to make in vivo because of their toxicity, complexity, and/or unusual cofactor requirements.

In certain exemplary embodiments, methods of making an in vitro assembled ribosomal subunit and/or ribosome are provided. In certain aspects, a modular, step-wise approach is provided in which in vivo purified portions of ribosomes and/or in vitro produced purified portions of ribosomes can be used to make natural ribosomes or ribosomal subunits, semi-synthetic ribosomes or ribosomal subunits (i.e., portions are in vivo purified and portions are in vitro produced (i.e., by in vitro transcription and/or in vitro translation)) as well as fully synthetic ribosomes or ribosomal subunits (i.e., the entire ribosome or ribosomal subunit is made up of portions that were in vitro produced (i.e., by in vitro transcription and/or in vitro translation)). As used herein, a portion of a ribosome refers to a polypeptide, a ribosomal subunit or an rRNA that can be used to produce a ribosome. Proteins and/or polypeptides produced by in vitro translation are referred to herein as “synthetic proteins” and “synthetic polypeptides,” respectively. In vitro transcribed rRNA is referred to herein as “synthetic rRNA.”

In certain aspects, ribosomal subunit assembly and/or ribosome assembly and in vitro rRNA transcription are performed in the same vessel, optionally concomitantly. In other aspects, ribosomal subunit assembly and/or ribosome assembly and in vitro translation are performed in the same vessel optionally concomitantly. In still other aspects, ribosomal subunit assembly and/or ribosome assembly, in vitro rRNA transcription, and in vitro translation are performed in the same vessel optionally concomitantly.

In certain exemplary embodiments, one or more of the methods described herein are performed in a vessel, e.g., a single, vessel. The term “vessel,” as used herein, refers to any container suitable for holding on or more of the reactants (e.g., for use in one or more transcription, ribosomal subunit/ribosome assembly, and/or translation steps) described herein. Examples of vessels include, but are not limited to, a microtitre plate, a test tube, a microfuge tube, a beaker, a flask, a multi-well plate, a cuvette, a flow system, a microfiber, a microscope slide and the like.

In certain exemplary embodiments, physiologically compatible (but not necessarily natural) ions and buffers are utilized for coupled ribosome assembly and translation, e.g., potassium glutamate, ammonium chloride and the like. Ribosomal subunits are reconstituted in physiological conditions (e.g., constant temperature and magnesium). Using cytoplasmic mimicry as a guide, salt conditions are provided as well as salts themselves in which ribosomal subunits are reconstituted. Physiological cytoplasmic salt conditions are well-known to those of skill in the art.

In certain exemplary embodiments, methods for the in vitro assembly of ribosomes and/or ribosomal subunits are provided. As used herein, the term assemble refers to the ability of portions of ribosomes to interact with one another. As used herein, the terms “bind,” “binding,” “interact,” “interacting,” “occupy” and “occupying” refer to covalent interactions, noncovalent interactions and steric interactions. A covalent interaction is a chemical linkage between two atoms or radicals formed by the sharing of a pair of electrons (a single bond), two pairs of electrons (a double bond) or three pairs of electrons (a triple bond). Covalent interactions are also known in the art as electron pair interactions or electron pair bonds. Noncovalent interactions include, but are not limited to, van der Waals interactions, hydrogen bonds, weak chemical bonds (via short-range noncovalent forces), hydrophobic interactions, ionic bonds and the like. A review of noncovalent interactions can be found in Alberts et al., in Molecular Biology of the Cell, 3d edition, Garland Publishing, 1994. Steric interactions are generally understood to include those where the structure of the compound is such that it is capable of occupying a site by virtue of its three dimensional structure, as opposed to any attractive forces between the compound and the site.

In certain exemplary embodiments, one or more reporter polypeptides and/or proteins are utilized as a read-out to assay ribosomal subunit and/or ribosome activity (i.e., the ability of the ribosomal subunit and/or ribosome to mediate translation). In certain aspects, the polypeptide and/or protein contains a detectable label. In other aspects, the reporter polypeptide and/or protein provides a biological activity (e.g., an enzymatic activity, bioluminescence, fluorescence or the like) that serves as a detectable label.

Examples of detectable labels include various radioactive moieties, enzymes, prosthetic groups, fluorescent markers, luminescent markers, bioluminescent markers, metal particles, protein-protein binding pairs, protein-antibody binding pairs and the like. Examples of fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases and the like. Identifiable markers also include radioactive compounds such as ¹²⁵I, ³⁵S, ¹⁴C, or ³H. Identifiable markers are commercially available from a variety of sources.

Embodiments of the present invention are based on the use of multiplex automated genome engineering (MAGE) (See U.S. Patent Application No. 2009/0317910, hereby incorporated by reference in its entirety) to introduce tags (e.g., his-tags or other tags such as flag-tag, avidin-binding peptides and the like) into endogenous copies of translation factors to reduce number of strains needed to purify factors, and/or improve yield of factors by taking advantage of the native overproduction of these proteins. Examples of proteins for in vivo and/or in vitro overproduction using the proteins-synthetic (or self-replicating) system of the present invention include (but are not limited to): dnaK, rpsT, ileS, frr, tilS, proS, cysS, leuS, miaB, glnS, infA, serS, rpsA, asnS, rlmL, mnmA, tyrS, aspS, argS, metG, mnmC, gltX, hisS, rlmN, iscS, tadA, rluD, rpsP, alaS, rpsU, rpsO, infB, fmt, rplQ, trpS, mnmE, mnmG, miaA, rpsF, valS, prfC, rpsB, tsf, prfA, prmC, rplS, trmD, lysS, prfB, rpmA, rplU, rpsl, rplM, glyS, glyQ, groS, groL, rpsR, rpll, rpsD, rpsK, rpsM, rpmJ, pheT, pheS, pheM, rplT, rpml, infC, thrS, rplO, rpmD, rpsE, rplR, rplF, rpsH, rpsN, rplE, rplX, rplN, rpsQ, rpmC, rplP, rpsC, rplV, rpsS, rplB, rplW, rplD, rplC, rpsJ, tufA, fusA, rpsG, rpsL, rplK, rplA, rplJ, rplL

In certain exemplary embodiments, vectors such as, for example, expression vectors, containing a nucleic acid encoding one or more rRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the invention is intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more rRNAs or reporter polypeptides and/or proteins described herein) in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner which allows for expression of the nucleotide sequence (e.g., in an in vitro ribosomal assembly, transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990).

As used herein, the term “amino acid” includes organic compounds containing both a basic amino group and an acidic carboxyl group. Included within this term are natural amino acids (e.g., L-amino acids), modified and unusual amino acids (e.g., D-amino acids and β-amino acids), as well as amino acids which are known to occur biologically in free or combined form but usually do not occur in proteins. Natural protein occurring amino acids include alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline, and valine. Natural non-protein amino acids include arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5,-triiodothyronine, and 3,3′,5,5′-tetraiodothyronine. Modified or unusual amino acids include D-amino acids, hydroxylysine, 4-hydroxyproline, N-Cbz-protected amino acids, 2,4-diaminobutyric acid, homoarginine, norleucine, N-methylaminobutyric acid, naphthylalanine, phenylglycine, α-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

As used herein, the term “peptide” includes compounds that consist of two or more amino acids that are linked by means of a peptide bond. Peptides may have a molecular weight of less than 10,000 Daltons, less than 5,000 Daltons, or less than 2,500 Daltons. The term “peptide” also includes compounds containing both peptide and non-peptide components, such as pseudopeptide or peptidomimetic residues or other non-amino acid components. Such compounds containing both peptide and non-peptide components may also be referred to as a “peptide analog.”

As used herein, the term “protein” includes compounds that consist of amino acids arranged in a linear chain and joined together by peptide bonds between the carboxyl and amino groups of adjacent amino acid residues.

The term “nucleoside,” as used herein, includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al., Exp. Opin. Ther. Patents, 6: 855-870 (1996); Mesmaeker et al., Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides comprising probes or primers usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

Usually nucleosidic monomers are linked by phosphodiester bonds. Whenever an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al., Molecular Cloning, Second Edition (Cold Spring Harbor Laboratory, New York, 1989), and like references. Oligonucleotides and polynucleotides may be single stranded or double stranded.

Oligonucleotides and polynucleotides may optionally include one or more non-standard nucleotide(s), nucleotide analog(s) and/or modified nucleotides. Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, tables, and accompanying claims.

### Example I

### Reconstituted Ribosomal Subunits

Experiments were performed to determine whether ribosomes could be reconstituted from their native parts, and whether reconstituted ribosomes could be used to make proteins. Ribosomes were reconstituted according to the methods presented herein. The function of reconstituted ribosomes was tested in an S150 crude extract-based combined transcription and translation system. A salt environment was used that mimicked the cytoplasmic environment. S30 crude extract systems were also used (Jewett, M. C., & Swartz, J. R. (2004) Mimicking the Escherichia coli cytoplasmic environment activates long-lived and efficient cell-free protein synthesis. Biotechnol Bioeng 86, 19-26). The S150 system was based on the PANOx-SP cell-free protein synthesis (CFPS) platform. The physicochemical environment of the PANOx-Sp system is a significant break from previous translation systems used to assess ribosome activity (Nierhaus, Supra). To initiate protein synthesis, a DNA template, T7 RNA polymerase, energy substrates, nucleotides, and salts to mimic the cytoplasm were incubated with S150 extract and ribosomal subunits. To quantify the amount of synthesized protein, an activity assay was used (One-GLOW system from Promega).

Natural ribosomal RNA (rRNA) and total protein (TP) mixtures from E. coli were prepared from 30S and 50S subunits (Nierhaus K H (1990) Reconstitution of ribosomes, in Ribosomes and Protein Synthesis, A Practical Approach, Spedding, G., Editor Oxford University Press, Oxford, 161-189) derived from tightly coupled 70S ribosomes (Blanchard S C, Gonzalez R L, Kim H D, Chu S, Puglisi J D “Nat Struct Mol Biol” 2004; 11:10:1008-14). Initially, standard, non-physiological reconstitution procedures were used (Nierhaus, Supra). The classical 30S reconstitution is a one-step procedure requiring mixing of all components of the subunit (TP30 and 16S rRNA) in 20 mM Tris-HCl (pH 7.4 at 37° C.), 20 mM Mg²⁺ acetate, 400 mM NH₄Cl, 1 mM EDTA, 5 mM 2-mercaptoethanol at 42° C. for 20 minutes. Id. The 50S reconstitution is a two-step procedure. The components of the subunit (total protein from the 50S subunit (TP50), 5S rRNA, and 23S rRNA) are first mixed in 20 mM Tris-HCl (pH 7.4 at 37° C.), 4 mM Mg²⁺ acetate, 400 mM NH₄Cl, 0.2 mM EDTA, 5 mM 2-mercaptoethanol at 44° C. for 20 minutes. Then, the Mg²⁺ concentration is raised to 20 mM and the temperature raised to 50° C. for 90 minutes. Id.

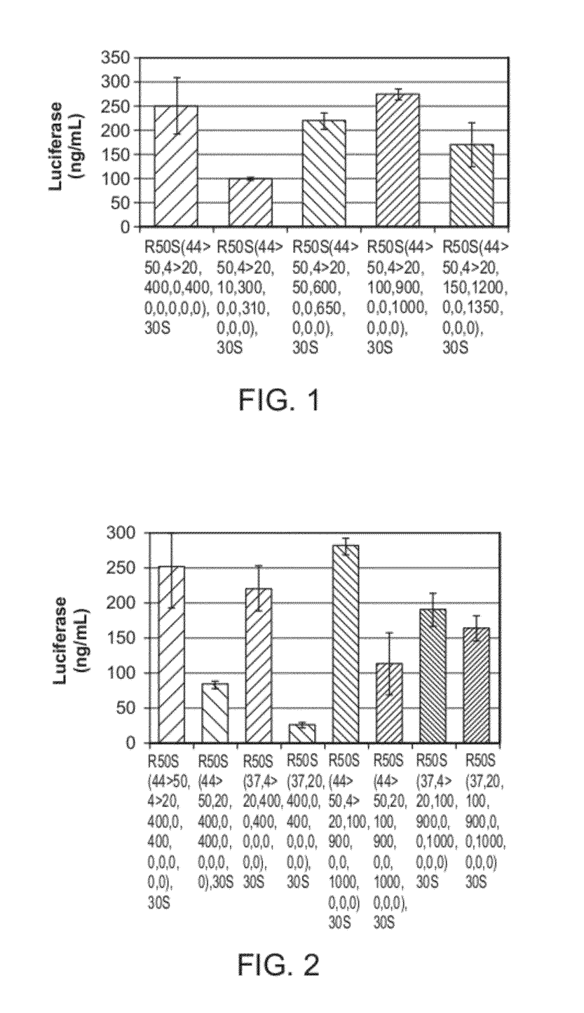

According to embodiments of the present invention, a one-step method is provided for reconstituting 30S and 50S subunits using the following: 20 mM magnesium, 0.2 mM EDTA, 20 mM Tris-acetate pH 7.4, 6 mM 2-mercaptoethanol, 400 mM potassium glutamate, 100 mM ammonium glutamate (FIG. 1). (Order of the ions in the axis—(44>50 indicates shift of 44° C. to 50° C.) A range of potassium glutamate concentrations could be used. FIG. 2 depicts that reconstituted ribosomes could be reconstituted at constant temperature and magnesium concentration (far right), purple indicates the same ions as depicted in FIG. 1.

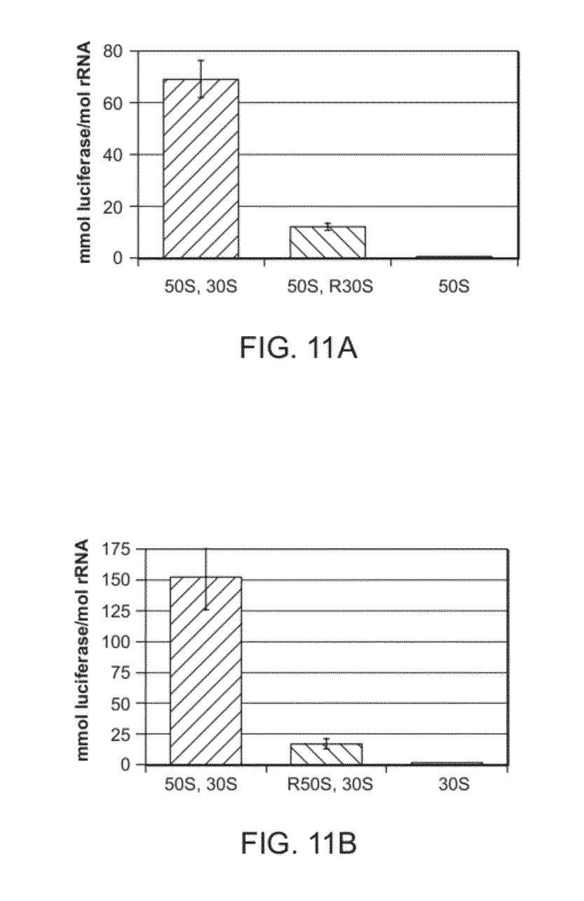

It was determined that reconstituted 30S ribosomal subunits combined with native 50S subunits could translate active reporter protein (i.e., luciferase) (FIG. 11A). Similarly, reconstituted 50S ribosomal subunits could be combined with native 30S subunits to translate active luciferase (FIG. 11B).

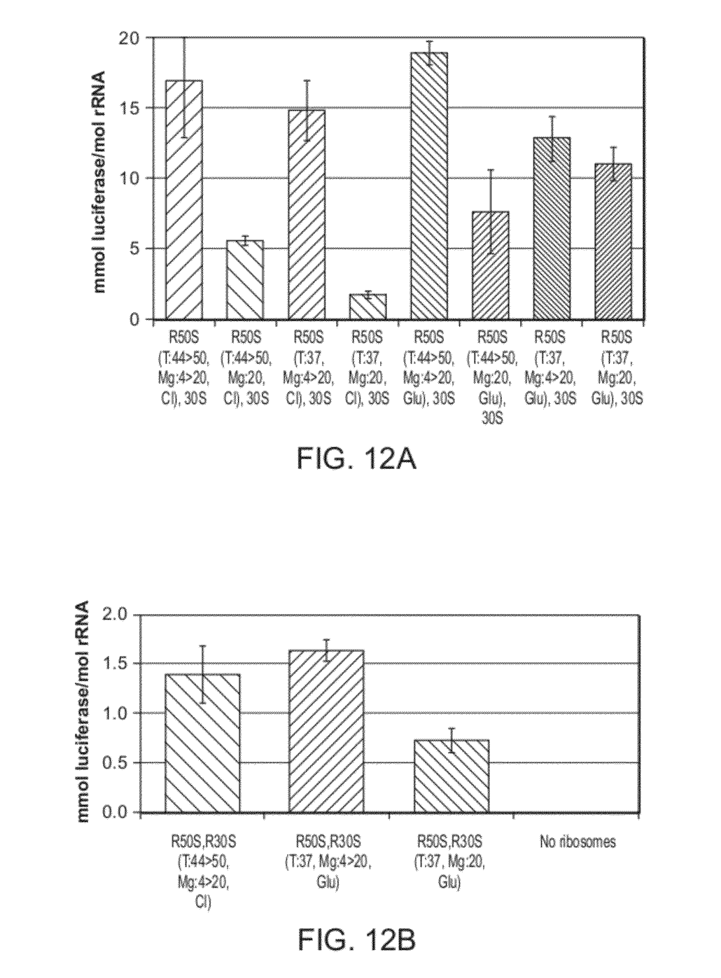

Experiments were next performed to determine whether ribosomal subunit reconstruction could be altered to be more physiologically relevant (e.g., to determine whether cytoplasmic mimicry could encourage more natural processes). Various conditions such as reconstitution times, temperatures, Mg²⁺ concentrations and the like were investigated. 50S ribosomal subunits could be reconstituted at 37° C. under conditions that mimic the cytoplasm (FIG. 12A). Reconstituted 70S ribosomes were obtained that could translate active luciferase protein (FIG. 12B).

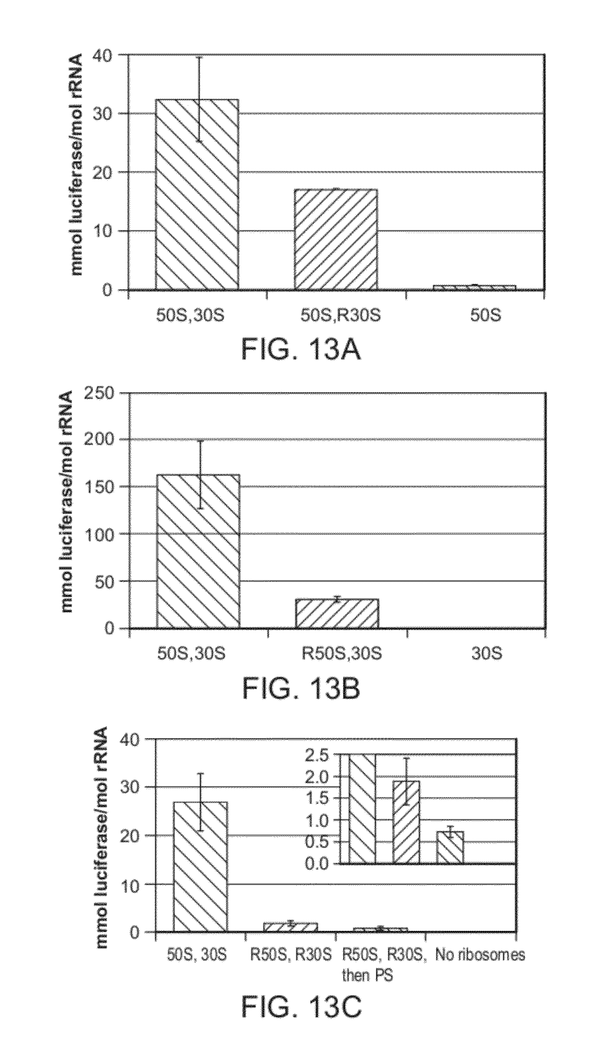

Experiments were performed to determine whether ribosomes could be reconstituted and synthesize protein within the same compartment, that is, whether self-assembly, transcription and translation could occur in the same compartment. 30S, 50S and 70S ribosomal subunits could each be reconstituted and synthesize luciferase in a single compartment (FIGS. 13A-13C, respectively).

### Example II

### Self-Assembly of Ribosomes from In Vitro-Transcribed rRNA

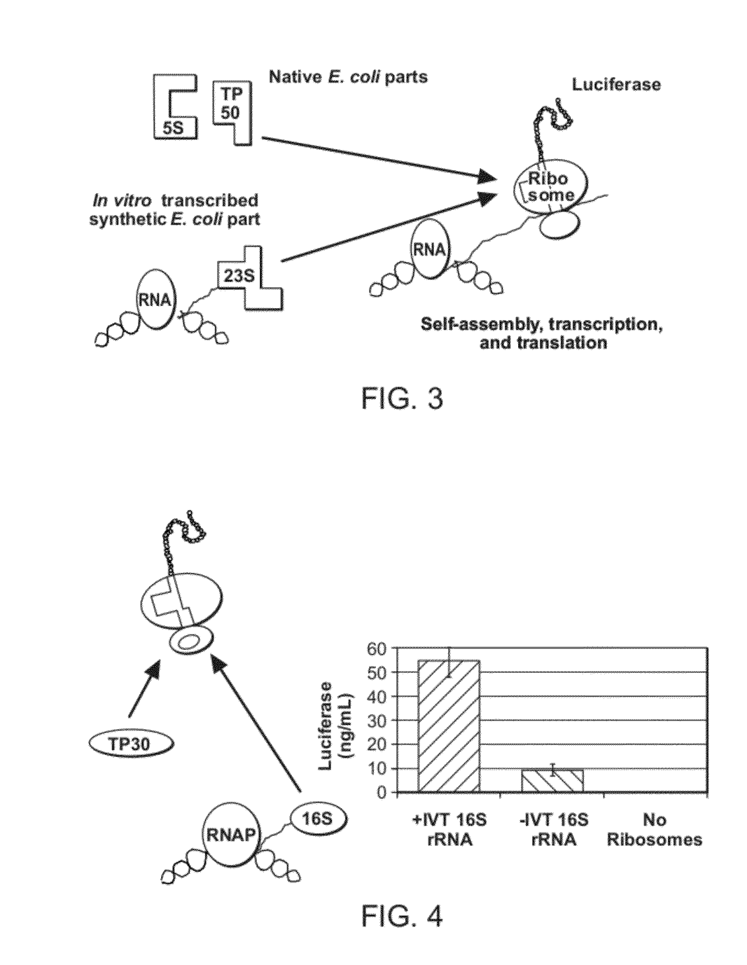

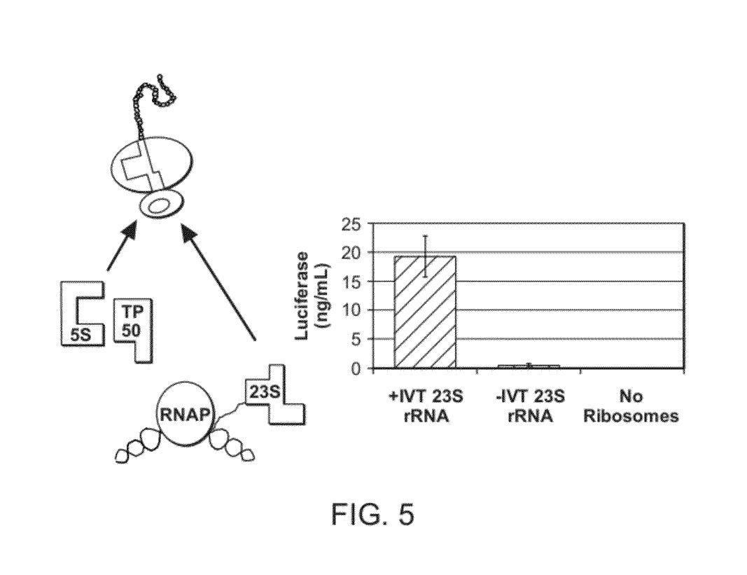

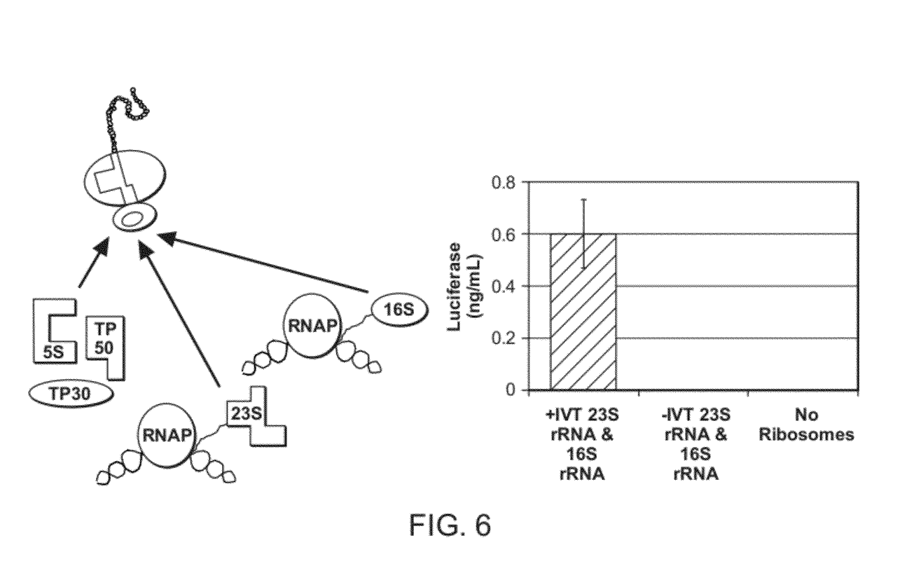

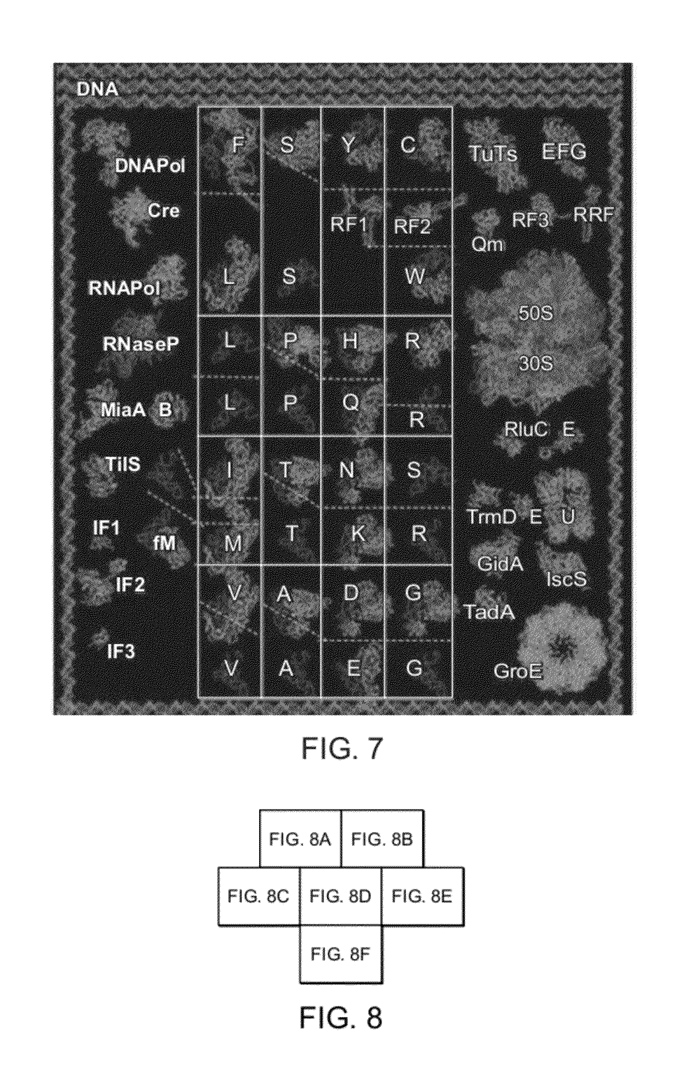

The following conditions were used for the translation system: PANOx-SP CFPS reactions were, in general, carried out in 1.5 ml Eppendorf tubes at 37° C. The standard reaction mixture contained the following components: 1.2 mM ATP, 0.85 mM each of GTP, UTP and CTP, 34 μg/ml folinic acid, 170.6 mug/ml E. coli tRNA mixture (Roche, Indianapolis, Ind.), 13.3 mug/ml pk7LUC plasmid (encoding the gene for luciferase but it can be any reporter gene), 100 μg/ml T7 RNA polymerase, 33 mM phosphoenolpyruvate, 2 mM each of 20 unlabeled amino acids, 0.33 mM NAD, 0.26 mM CoA, 130 mM potassium glutamate, 10 mM ammonium glutamate, 14 mM magnesium glutamate, 1.5 mM spermidine, 1 mM putrescine, 4 mM sodium oxalate, 57 mM HEPES buffer pH 7.4 0.6 Units/mL pyruvate kinase, and 0.24 volume of S150 extract. Reaction volumes were 15 μl. Accordingly, one embodiment is directed to reconstitution/self-assembly of ribosomes in one compartment and with the cell-free protein synthesis reaction (see FIGS. 3-6). FIG. 7 depicts the components for creating a synthetic self-replicating ribosome. Proteins are shown in purple, RNA in red and DNA in blue. The components include 54 ribosomal proteins, as well as RNA based enzymes involved in protein production and other molecules that interact with ribosomes. Higher speed and accuracy can be achieved by adding a few extra genes (i.e., in addition to a minimal genome).

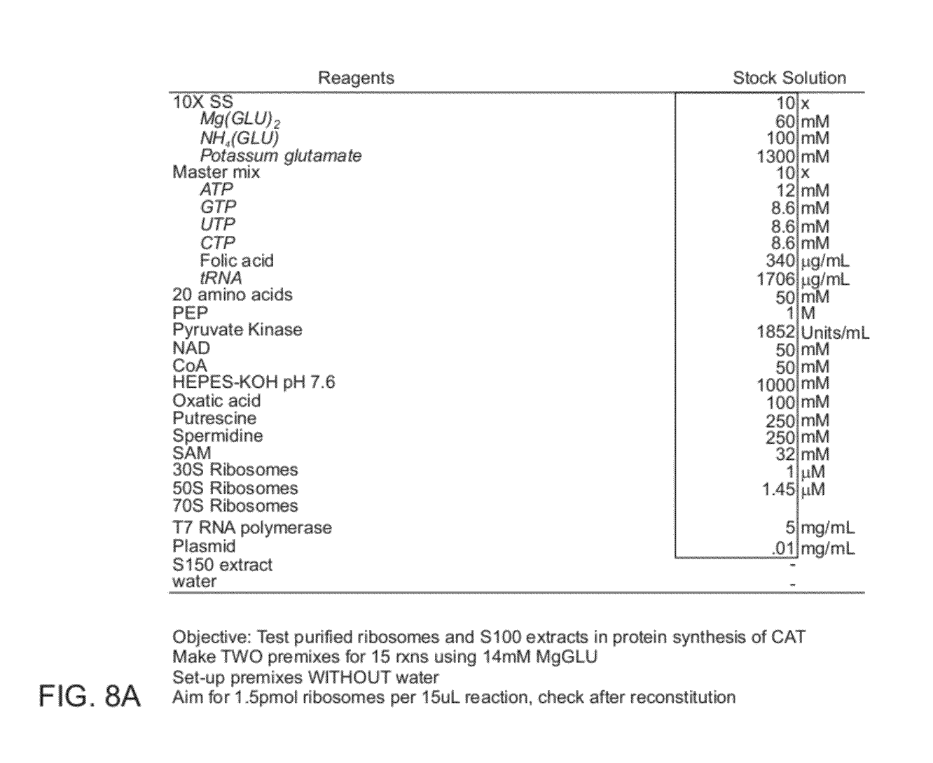

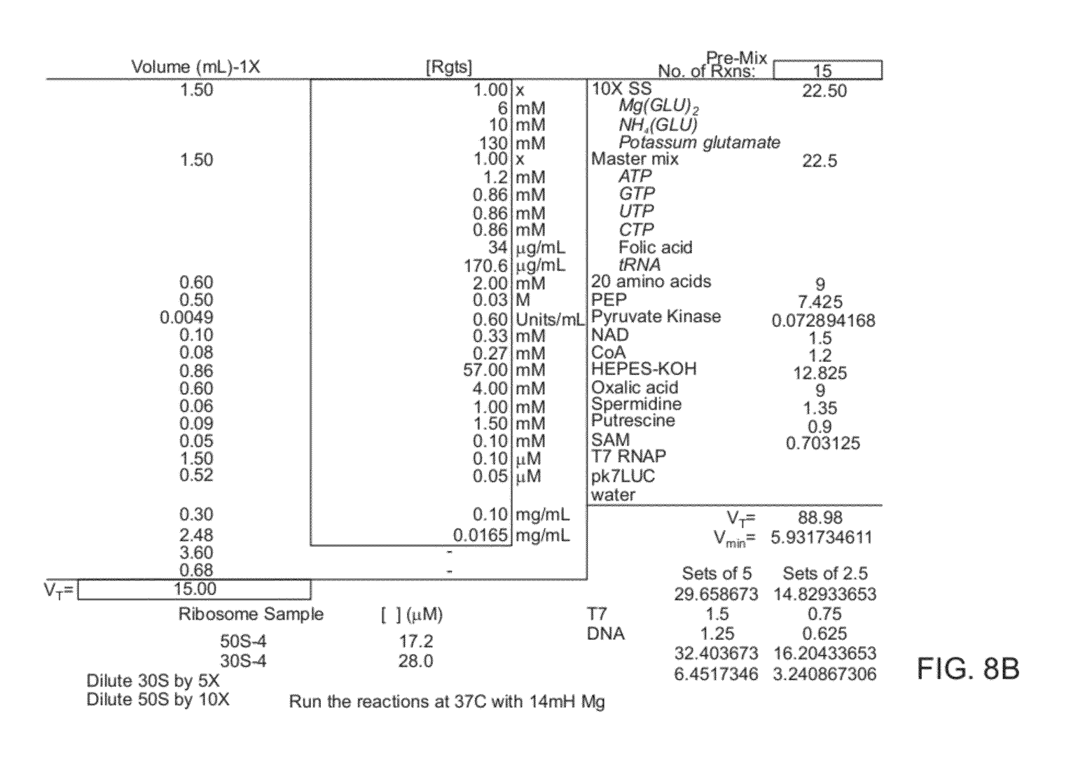

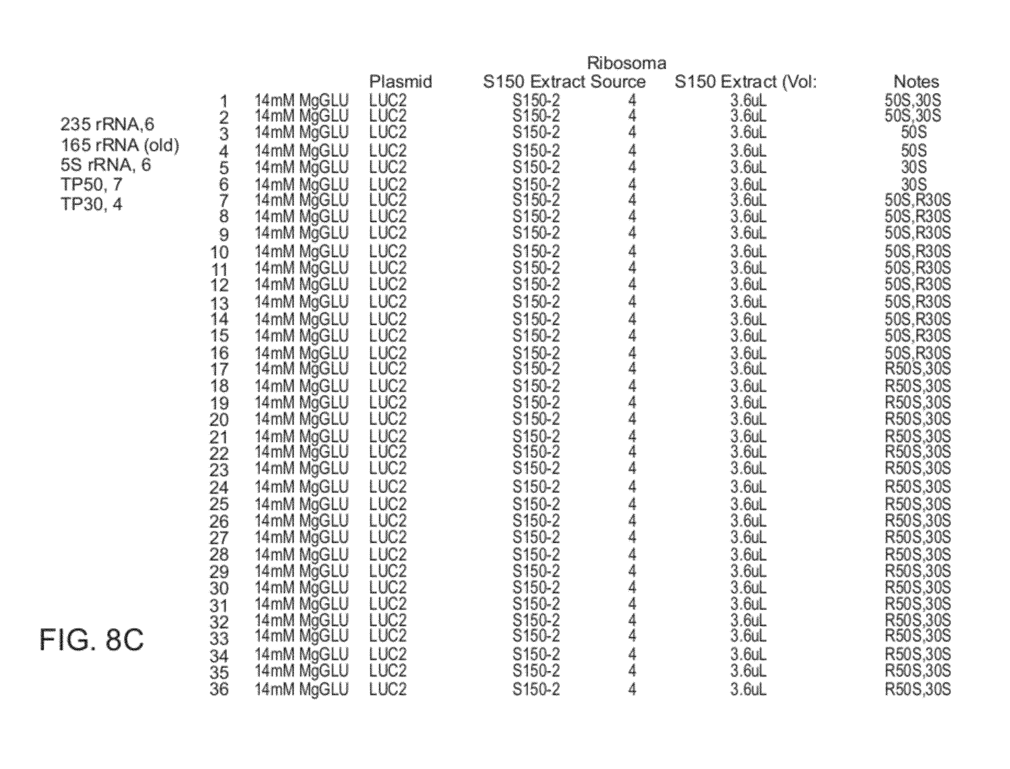

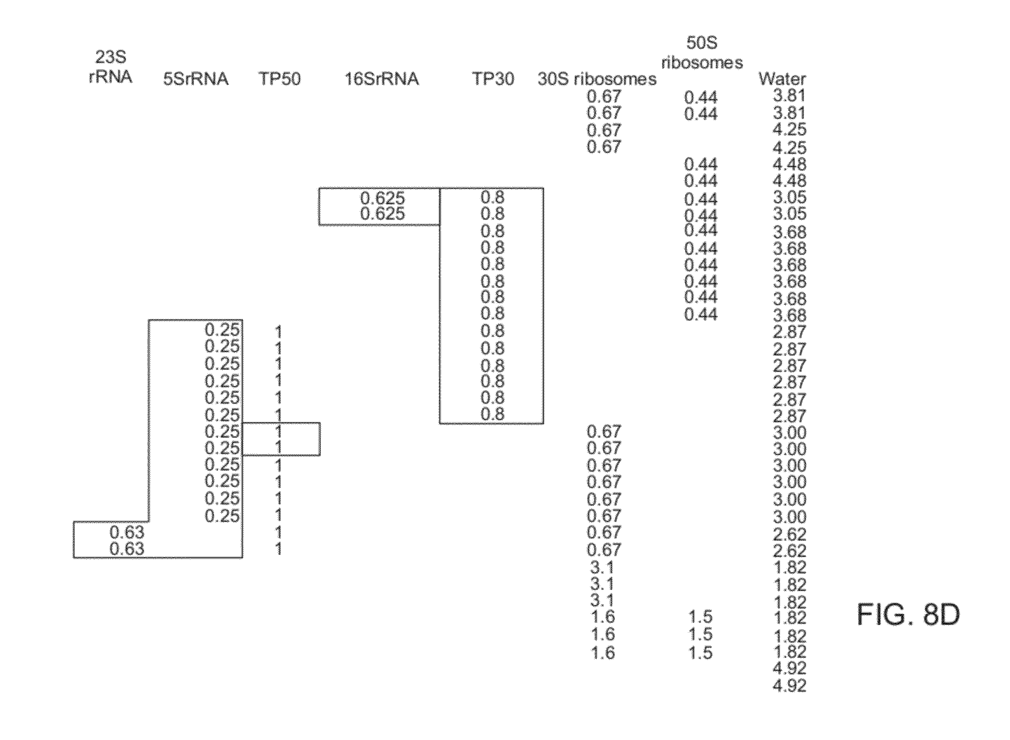

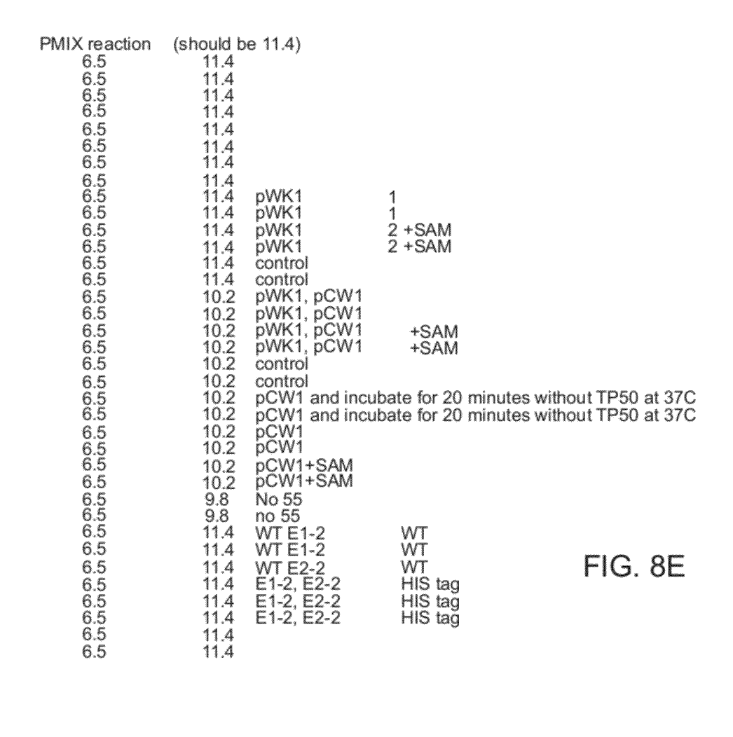

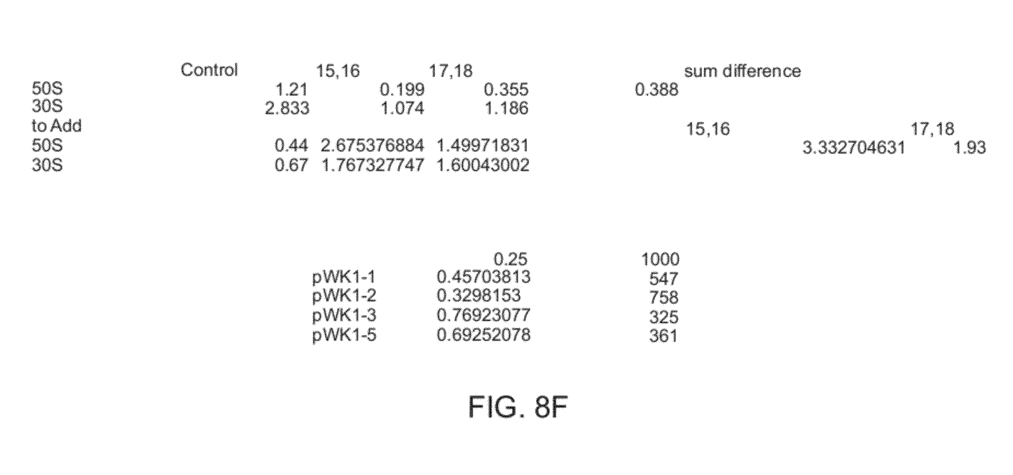

Embodiments of the present invention are further directed to the use of E. coli cell extracts and/or pure or partially pure proteins for modifying rRNA and/or facilitating rRNA folding and/or assembly. The crude extract facilitates in modifications of in vitro transcribed rRNA (encoded on a plasmid), which is subsequently assembled with native E. coli proteins. Experimental conditions are set forth in FIG. 8.

PANOx-SP CFPS (cell-free protein synthesis) reactions were, in general, carried out in 1.5 ml Eppendorf tubes at 37° C. The standard reaction mixture contained the following components: 1.2 mM ATP, 0.85 mM each of GTP, UTP and CTP, 34 μg/ml folinic acid, 170.6 μg/ml E. coli tRNA mixture (Roche, Indianapolis, Ind.), 13.3 μg/ml pk7LUC plasmid (encoding the gene for luciferase but it can be any reporter gene), 100 μg/ml T7 RNA polymerase, 33 mM phosphoenolpyruvate, 2 mM each of 20 unlabeled amino acids, 0.33 mM NAD, 0.26 mM CoA, 130 mM potassium glutamate, 10 mM ammonium glutamate, 14 mM magnesium glutamate, 1.5 mM spermidine, 1 mM putrescine, 4 mM sodium oxalate, 57 mM HEPES buffer pH 7.4 0.6 Units/mL pyruvate kinase, and 0.24 volume of S150 extract. Reaction volumes were 15 μl.

Embodiments of the present invention are directed to optional co-transcription and/or co-translation to aid folding and assembly of the ribosomes. For example, T7 RNA polymerase and synthetic and/or natural proteins can be used.

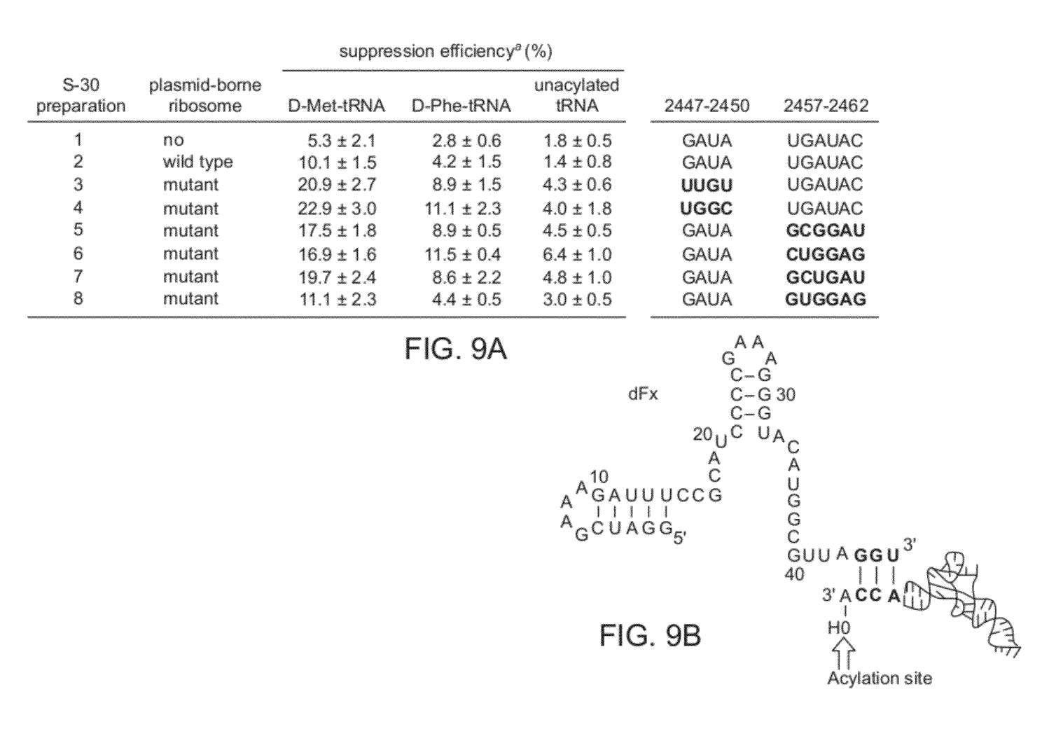

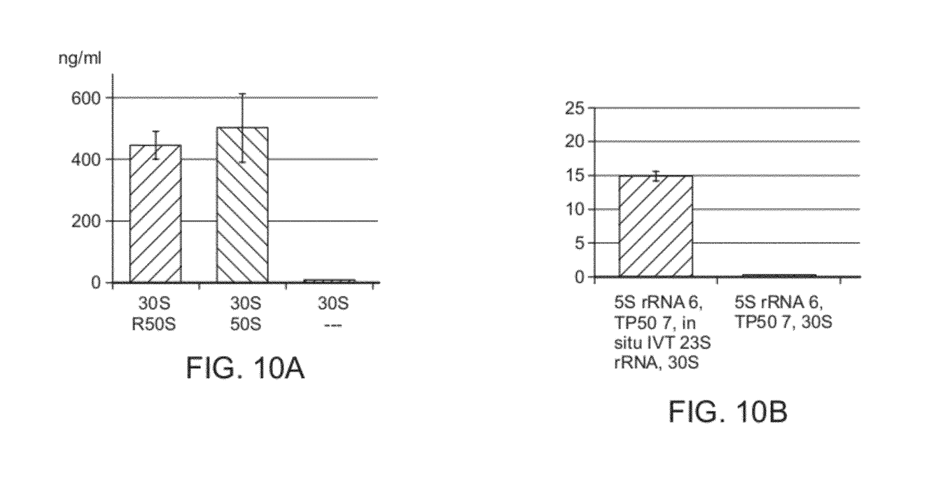

Embodiments of the present invention are further directed to the use of ribosome display of modified amino-acids (e.g. L-lysyl-biotin) to select for ribosomes which are more active under standard conditions or altered conditions including changes in pH, temperature, redox level, detergents, and the like. According to this embodiment, the methods use one or more of (a) emulsions (Tawfik, D. S, and A. D. Griffiths (1998) Nat. Biotechnol. 16(7):652), (b) liposomes (Noireaux & Libchaber A vesicle bioreactor as a step toward an artificial cell assembly Proc. Natl. Acad. Sci. USA 2004), (c) polymer interactions, (d) specific protein-protein, protein-RNA and/or RNA-RNA interactions or the like. Embodiments of the present invention are directed to the use of ribosome display of modified amino-acids (e.g. D-lysyl-biotin) to select for ribosomes which are more active as above but specifically using mirror image amino acids (FIGS. 9A and 9B). This method overcomes barriers at the AA-tRNA-synthetase and 23S rRNA levels. Embodiments of the present invention are also directed to the use of ribosome display to permit use of modified amino-acids which not compatible with native ribosomes, for example bulky fluorescent groups used in FRET pairs (e.g., FIG. 10).

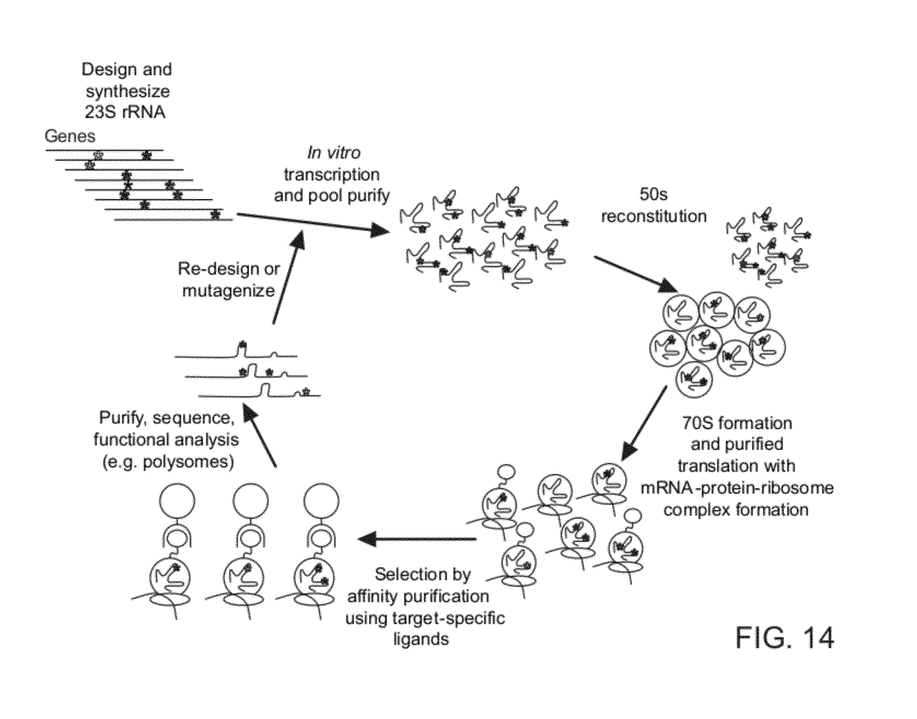

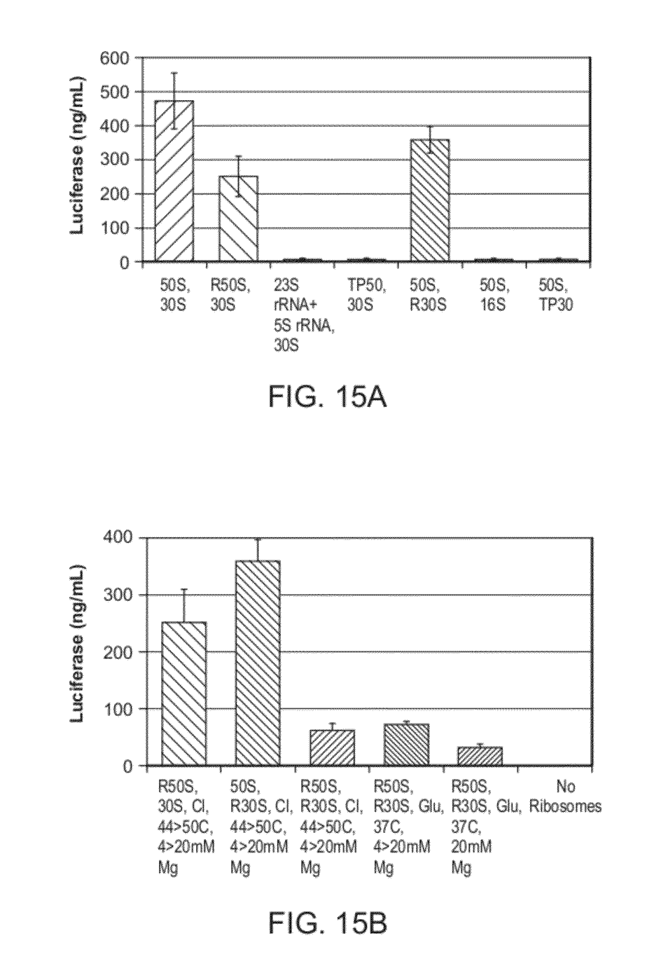

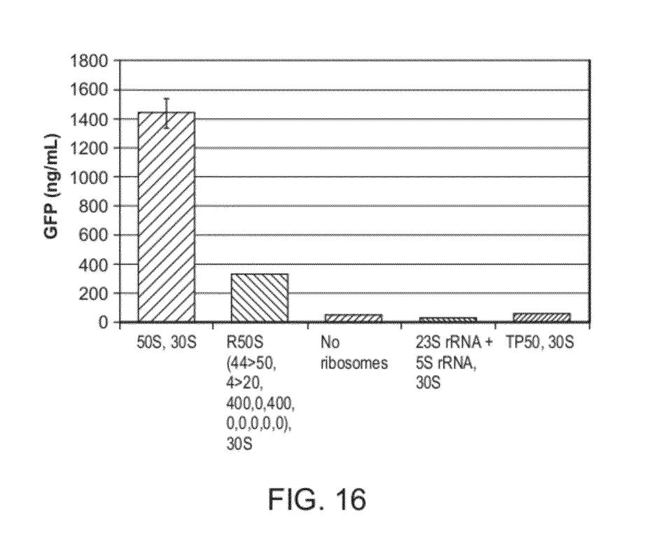

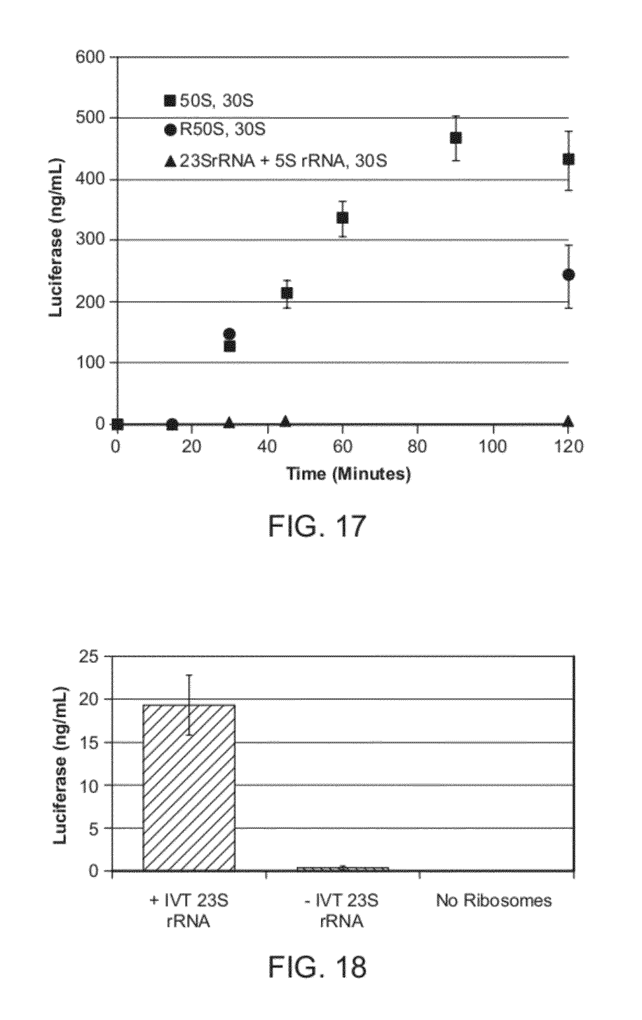

The strategy set forth in FIG. 14 was devised for isolating highly active, synthetic 23S rRNA. A variety of conditions were utilized to determine translation levels using reconstituted 50S and 30S ribosomal subunits (FIGS. 15A-15B and 16). Protein synthesis kinetics were determined for reconstituted 50S subunits (FIG. 17). In vitro transcribed 23S rRNA couples with self-assembly was determined to be able to generate functional ribosomes (FIG. 18).

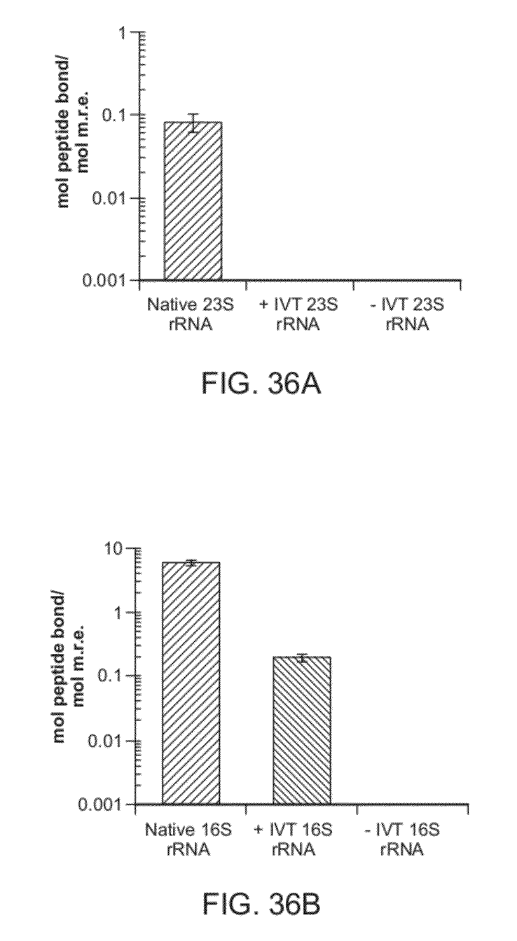

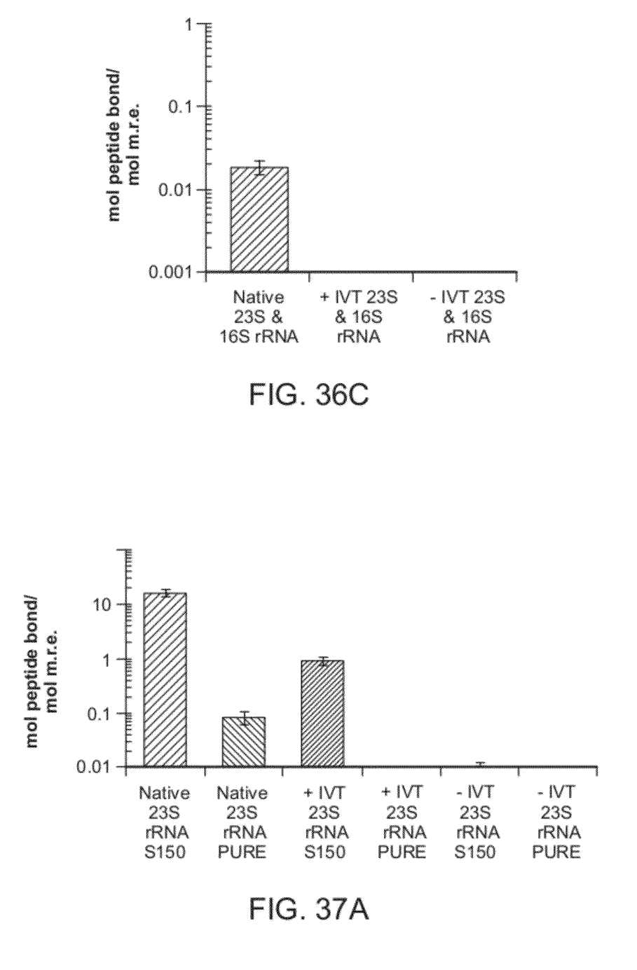

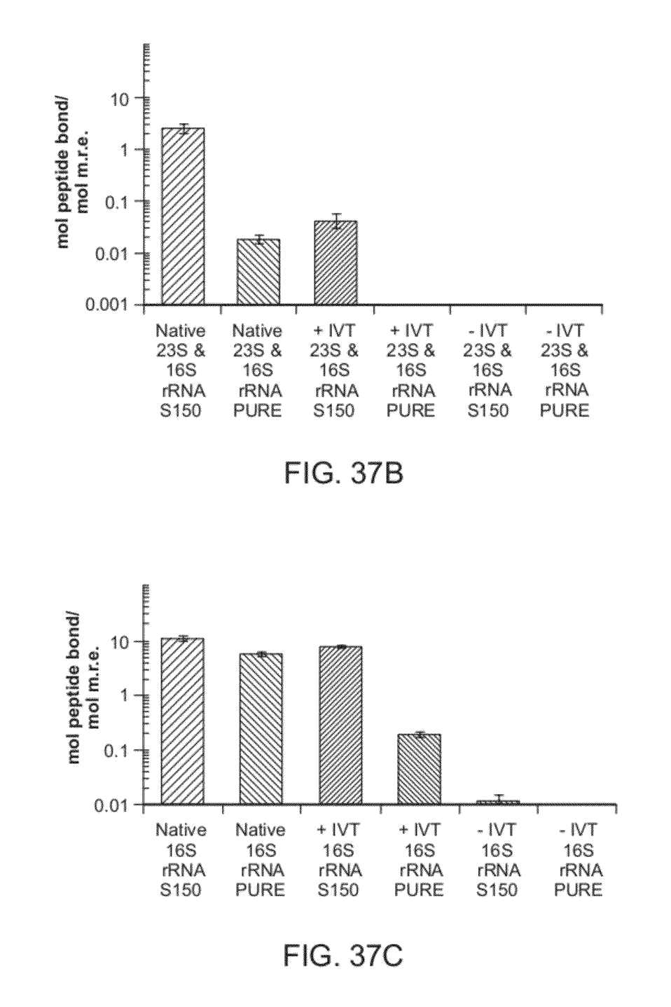

FIGS. 36A-36C depict ribosome synthesis and self-assembly using a PURE translation system (New England Biolabs, Beverly Mass.) lacking ribosomes instead of an S150 extract. FIGS. 37A-37C compare rRNA synthesis, ribosome self-assembly and protein synthesis between the PURE translation system and the S150 extracts. The PURE translation system was successful for 30 S subunits.

### Example III

### Discussion

Reconstituted ribosomes were generated that expressed full-length proteins, and ribosomes were reconstituted under physiological conditions. One compartment ribosome self-assembly was mimicked in vitro. Further, in vitro transcribed rRNA was incorporated into functional, synthetic ribosomes.

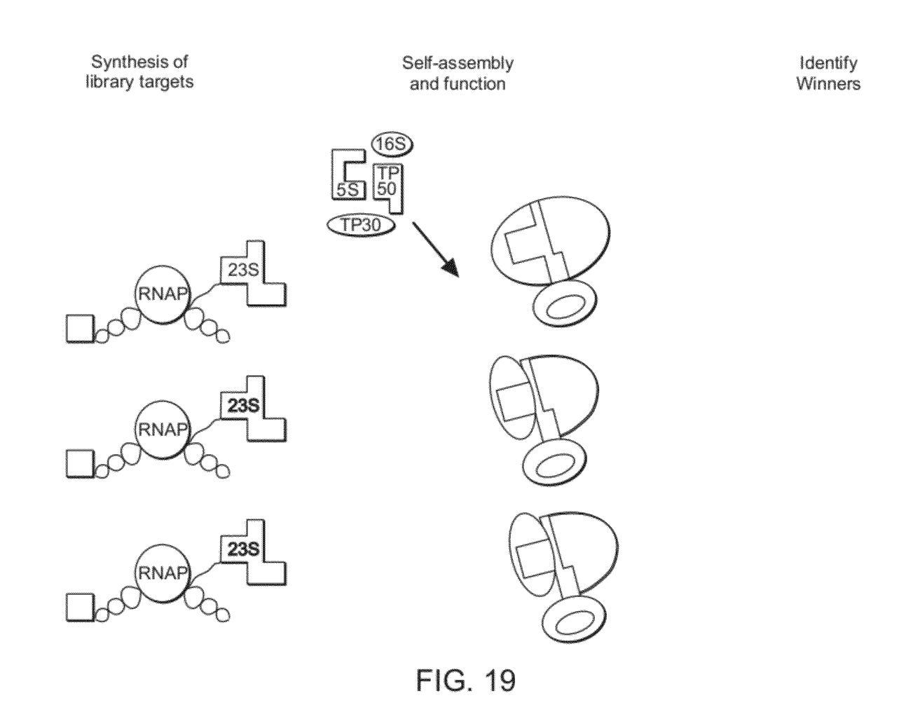

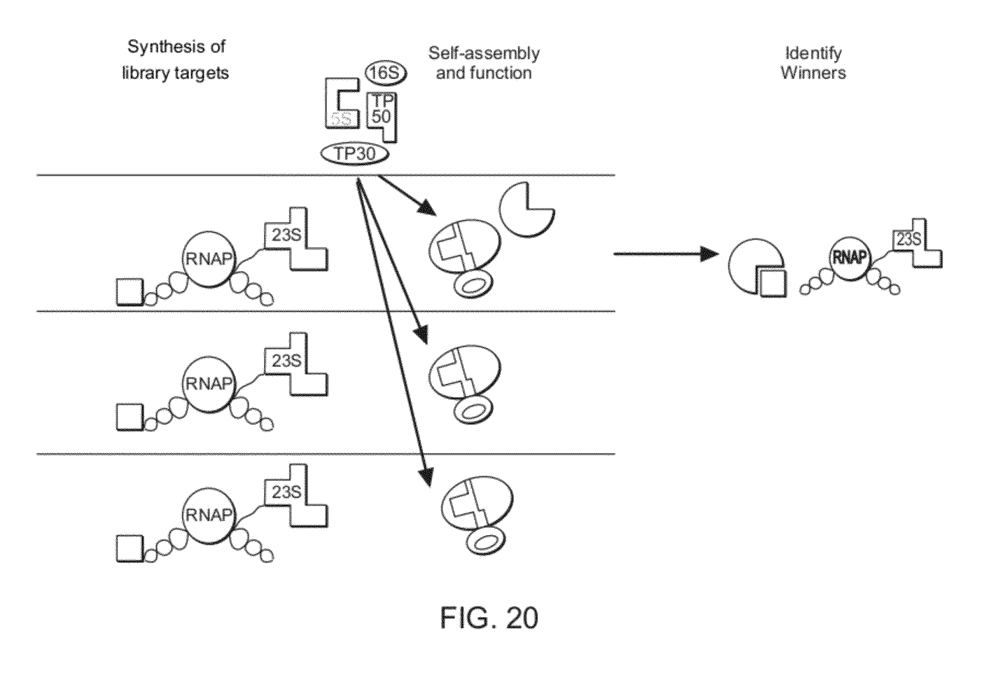

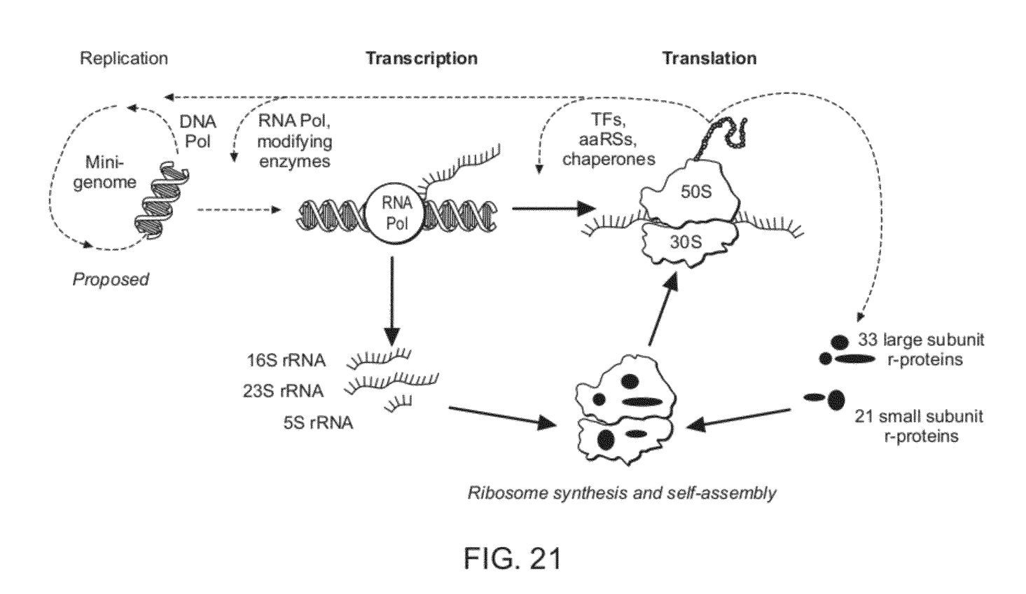

Further aspects of the present invention are directed to building the ribosomal protein genes required for in situ ribosome production, quantifying efficiencies of self-assembly, and developing selection strategies for evolving the ribosome. See also Forster and Church (2006) Molecular Systems Biology doi:10.1038/msb4100090. Strategies for synthesizing library targets and self-evolving ribosomes are depicted in FIGS. 19 and 20.

Synthetic ribosomes are useful for querying biological questions. They can be used to test our understanding of how life works and to generate useful biological systems useful in a variety of applications. According to certain aspects of the invention, synthetic ribosomes allow the production of non-natural proteins, for example, proteins containing D-amino acids. Synthetic ribosomes also allow the production of mirror-image proteins (e.g., peptidomimetic drugs that are resistant to breakdown by native enzymes). Further, D-amino acids are used for racemic crystallography wherein proteins of both chiralities are used to aid in crystallization of the protein.

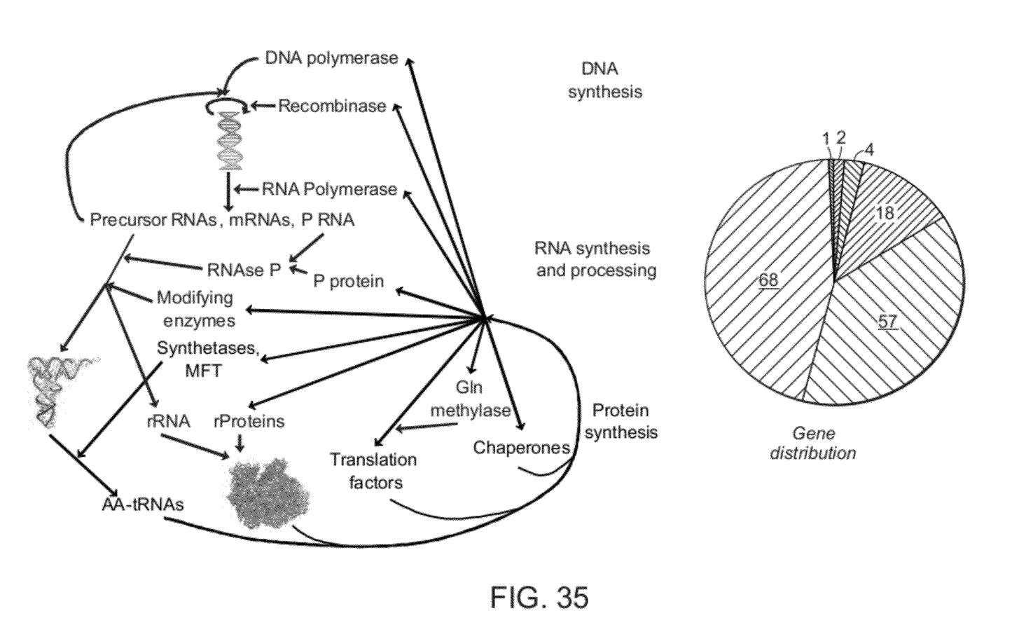

In one embodiment, the synthetic ribosomes described herein are useful for generating a synthetic replicon. The term “synthetic replicon” refers to a cell-free, self-replicating entity that carries out the biochemical activities of the “central dogma” (i.e., DNA→RNA→protein) from a minimal genome of approximately 150 genes derived from, for example, E. coli and its bacteriophages (See FIG. 35 and Forster and Church (2006) Molecular Systems Biology doi:10.1038/msb4100090). Synthetic replicons will provide a variety of advantages to researchers including, but not limited to: the ability to directly access and control the genome; it is close to a fully understood system; the cellular overhead is removed; it would enable system-by-system debugging; it can be used to test what is known and what is not known; and it provides flexibility for producing reagents (e.g., biopolymers, enzymes, pharmaceutical products and the like) that are difficult to make in vivo because of a variety of difficulties (e.g., toxicity, complexity, unusual co-factor requirements or the like). Synthetic replicons can be used in cell-free synthetic biology to generate useful, cost-effective factories for manufacturing therapeutics (e.g., human therapeutics) and valuable biochemicals that are difficult to make in vivo. Synthetic replicons reduce complexity, remove structural barriers and do not require cell viability.

### Polymerization Rates

The rate of protein synthesis using the ribosomes described herein relative to in vivo protein synthesis is as follows. Consider the 70S ribosomes reconstituted under physiological conditions (e.g., 37° C. and 20 mM Mg²⁺)

Luciferase Yield=25 ng/mL=400 μM (per one hour)

### Ribosomes=50 nM

### Luciferase/Ribosomes=0.008

However, some percentage of ribosomes are inactive. To carry out this calculation, one would need to know the number of active ribosomes, but this quantity is unknown. % ribosomes translating (MAX)=1%. Assuming one ribosome makes one luciferase protein in one hour, the amino acid polymerization rate is: 558 amino acids/1 hour, approximately 0.15 amino acids/second. Since the synthetic replicon needs approximately 40,000 amino acids, with only one ribosome, it would take approximately 72 hours to make.

### Example IV

In Vitro Integration of Ribosomal RNA Synthesis, Ribosome Self-Assembly and Protein Synthesis

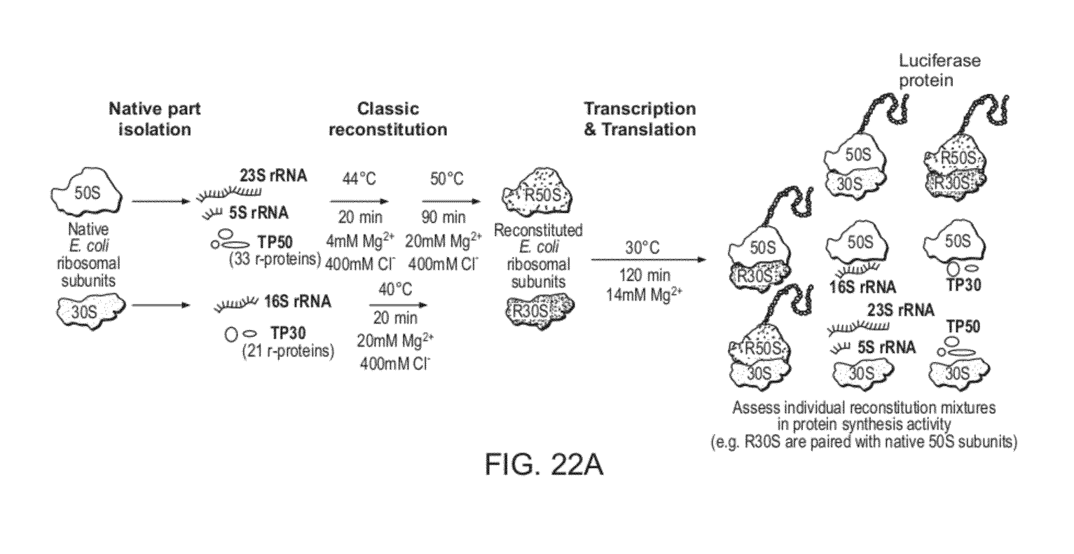

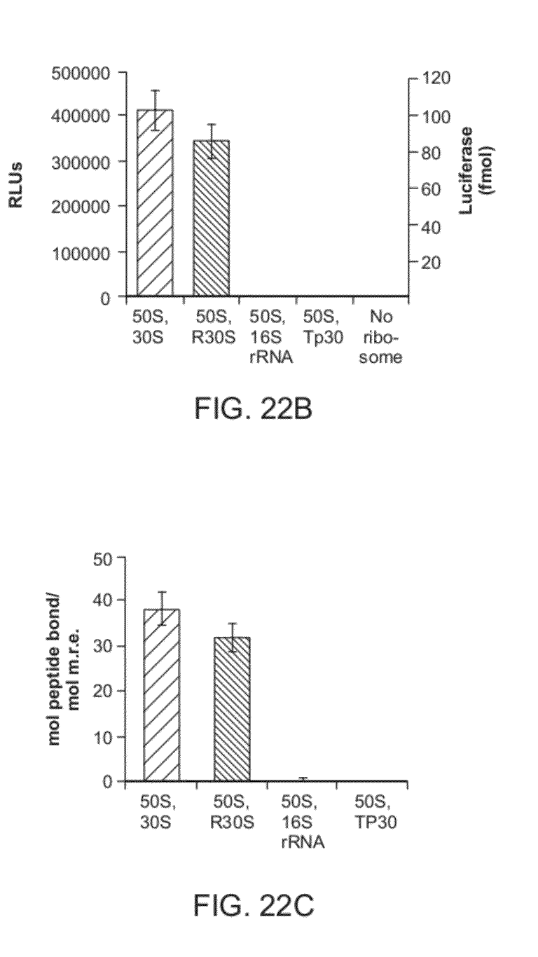

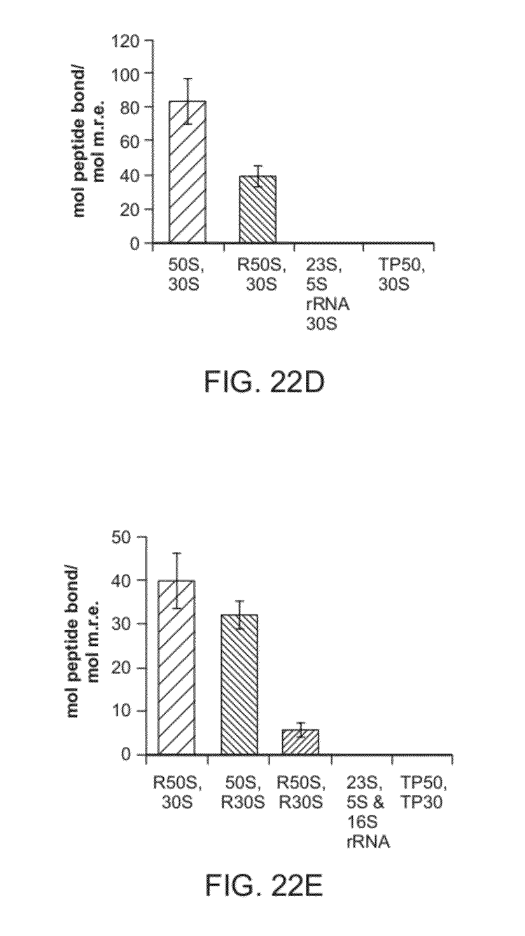

An attempt was made to synthesize active protein from ribosomal subunits reconstituted with native components. Natural rRNAs, total proteins from 30S subunits (TP30), and total proteins from 50S subunits (TP50) were isolated from the subunits of tightly-coupled 70S ribosomes (Nierhaus, K. H., Reconstitution of ribosomes, in Ribosomes and Protein Synthesis, A Practical Approach. (Oxford University Press, Oxford, 1990)). Following classical assembly with these components (FIG. 22A), ribosome reconstitution mixtures were added to an S150 crude extract transcription and translation system that closely mimics the E. coli cytoplasm (Jewett, M. C. & Swartz, J. R., Mimicking the Escherichia coli cytoplasmic environment activates long-lived and efficient cell-free protein synthesis. Biotechnol Bioeng 86, 19-26 (2004); Jewett, M. C., Calhoun, K. A., Voloshin, A., Wuu, J. J., & Swartz, J. R., An integrated cell-free metabolic platform for protein production and synthetic biology. Mol Syst Biol 4, 220 (2008)). S150 extract provides the necessary components for translation (e.g., aminoacyl-tRNA synthetases, translation factors, etc.), but lacks ribosomes. Reactions were incubated for 2 hours at 30° C. and the production of firefly luciferase (Fluc), a 550 amino acid, two-domain eukaryotic protein, was assessed by measuring luminescence. Luminescence was converted to an estimate of peptide bonds synthesized per maximum ribosome equivalents (m.r.e.=the maximum possible number of ribosomes in the reaction). The m.r.e. basis provided the most conservative assessment of protein synthesis activity of the methods described herein, because not all ribosomal parts are reconstituted into ribosomes, not all ribosomes are functionally active, truncated products are produced (including a known internal translation start site in Fluc), and not all synthesized Fluc is expected to be soluble and active.

In vitro assembled ribosomal subunits were capable of synthesizing complex proteins. When synthesizing Fluc, 30S and 50S reconstitution mixtures showed approximately 85% and approximately 50% of their native subunit activity, respectively (FIGS. 22B-22D), which is consistent with the yield of active particles determined using the poly(Phe) assay (Nierhaus, Supra). Negligible protein synthesis was observed with reconstitution mixtures having only 16S rRNA or TP30 (FIGS. 22B, 22C), or only 23S and 5S rRNA or TP50 (FIG. 22D), ruling out the possibility of contaminating species and confirming dependence on exogenously added ribosomal subunits. When added together, 50S and 30S reconstitution mixtures were also competent in protein synthesis (FIG. 22E). However, protein synthesis activity was approximately 15% of the native ribosomal activity, supporting a previous hypothesis that native subunits mask defects in reconstituted ribosomal particles when individually tested in polypeptide synthesis (Maki, J. A. & Culver, G. M., Recent developments in factor-facilitated ribosome assembly. Methods 36, 313-320 (2005)).

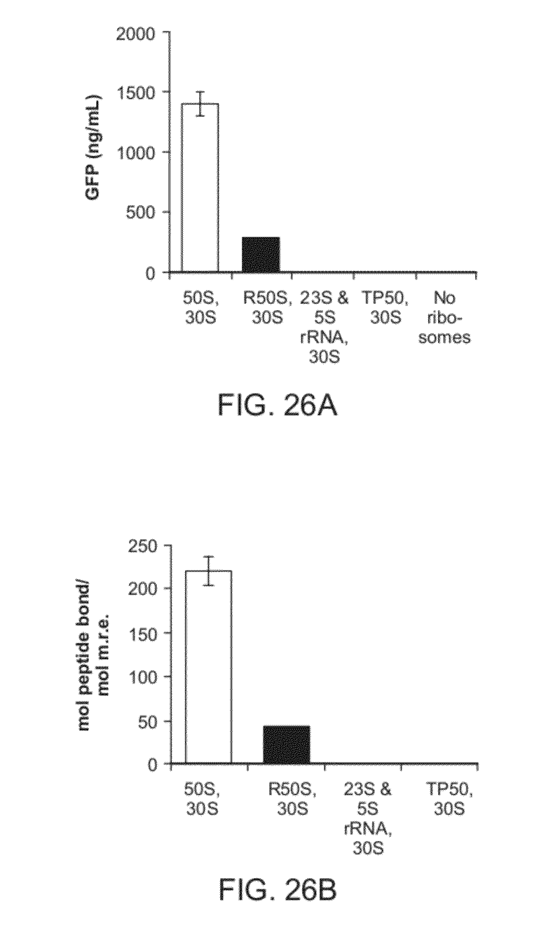

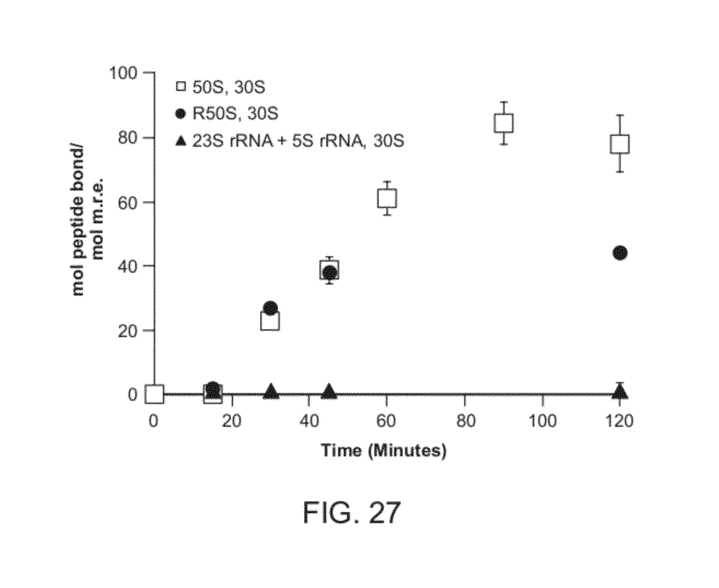

To test if protein synthesis activity was specific to Fluc, 50S reconstitution mixtures were assessed to determine if they could also synthesize green fluorescent protein (GFP). It was observed that protein synthesis activity from 50S reconstitution mixtures was similar when synthesizing GFP or Fluc (approximately 40-45 peptide bonds per m.r.e.), with higher activity from native subunits (approximately 1 GFP molecule per m.r.e. as compared to approximately 0.15 Fluc molecule per m.r.e.) (FIG. 26). The accumulation of Fluc from 50S reconstitution mixtures was also measured as a function of time (FIG. 27). Almost 85% of the total product was made between 15 and 45 minutes at a bulk rate of 0.02 amino acids/m.r.e./sec. Since approximately 0.07 Fluc molecules are produced per m.r.e., it was assumed that about 5% of the ribosome pool was translating active Fluc for an adjusted bulk rate of approximately 0.3 amino acids/m.r.e./sec compared with approximately 20 amino acids/ribosome/sec in vivo (Bremmer, H. D. & Dennis P. P., in Escherichia coli and Salmonella: Cellular and Molecular Biology. (ASM Press, Washington D.C., 1996)).

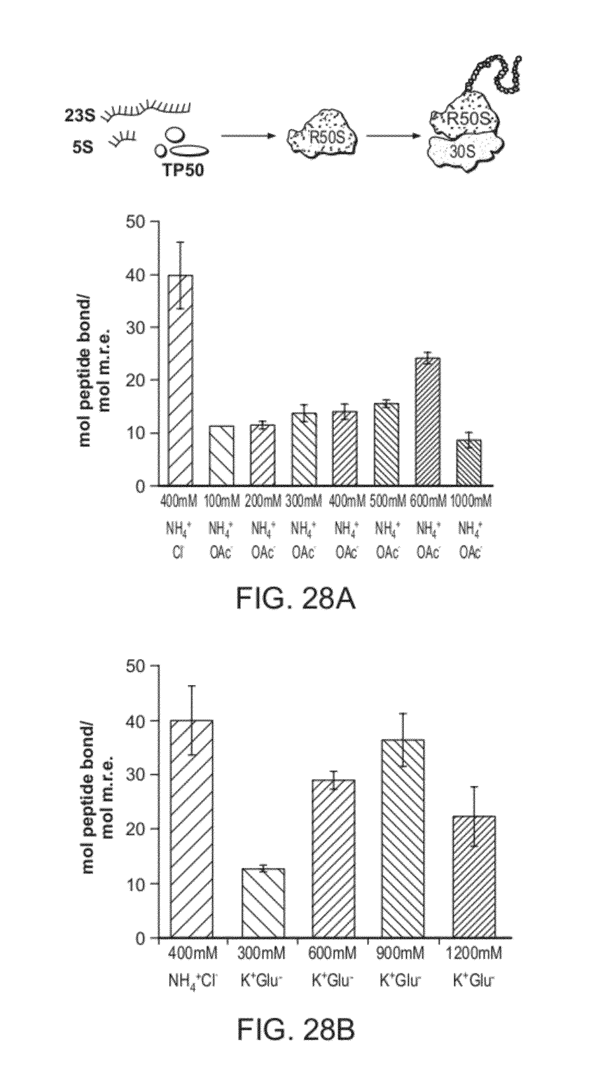

To make ribosome reconstitution more compatible with in vitro transcription and translation, three non-physiological conditions of the large subunit self-assembly protocol were altered: 1) the ionic environment, 2) the two-step high temperature incubation, and 3) the two-step magnesium shift. Changes in the ionic composition were first targeted because high chloride concentrations are non-physiological and may interfere with favorable non-covalent interactions (Record, M. T., Jr., Courtenay, E. S., Cayley, S., & Guttman, H. J., Biophysical compensation mechanisms buffering E. coli protein-nucleic acid interactions against changing environments. Trends Biochem Sci 23, 190-194 (1998)). 50S reconstitutions were carried out by substituting ammonium acetate (100-1000 mM) or potassium glutamate (300-1200 mM) for 400 mM NH₄Cl because glutamate and acetate are more predominant in the E. coli cytoplasm and less perturbing than chloride (having delocalized charge). Id. Additionally, glutamate is the preferred anionic species for in vitro systems and potassium is the most predominant cation in the cytoplasm. Id. Protein synthesis activity of 50S reconstitution mixtures assembled in ammonium acetate was, at best, 60% as active as subunits assembled in the classical conditions (FIG. 28A). In contrast, 50S reconstitution mixtures assembled in 900 mM potassium glutamate showed equivalent activity to those assembled in 400 mM NH₄Cl (FIGS. 23 and 28B).

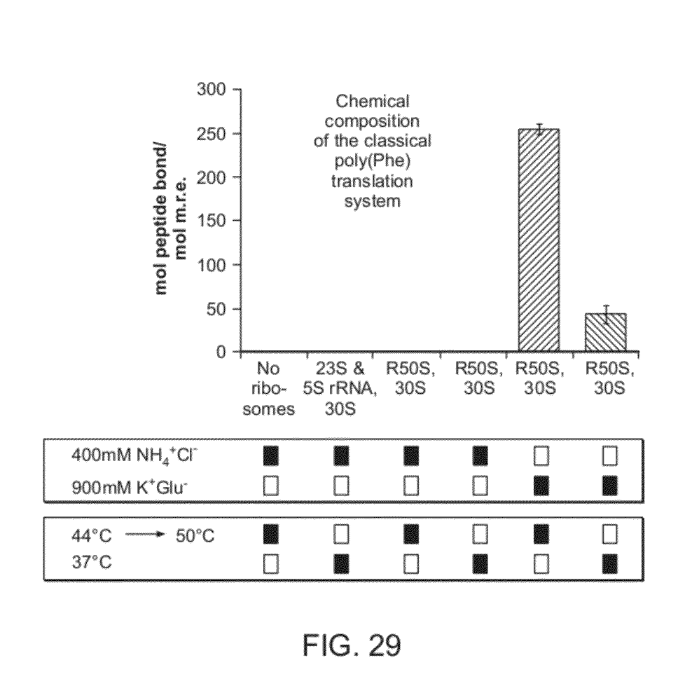

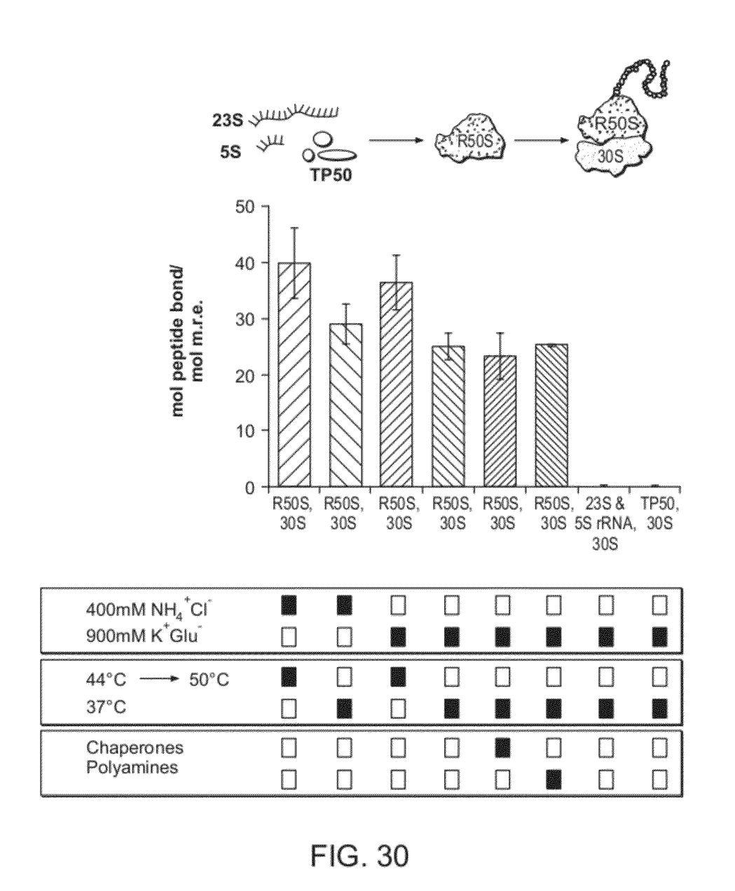

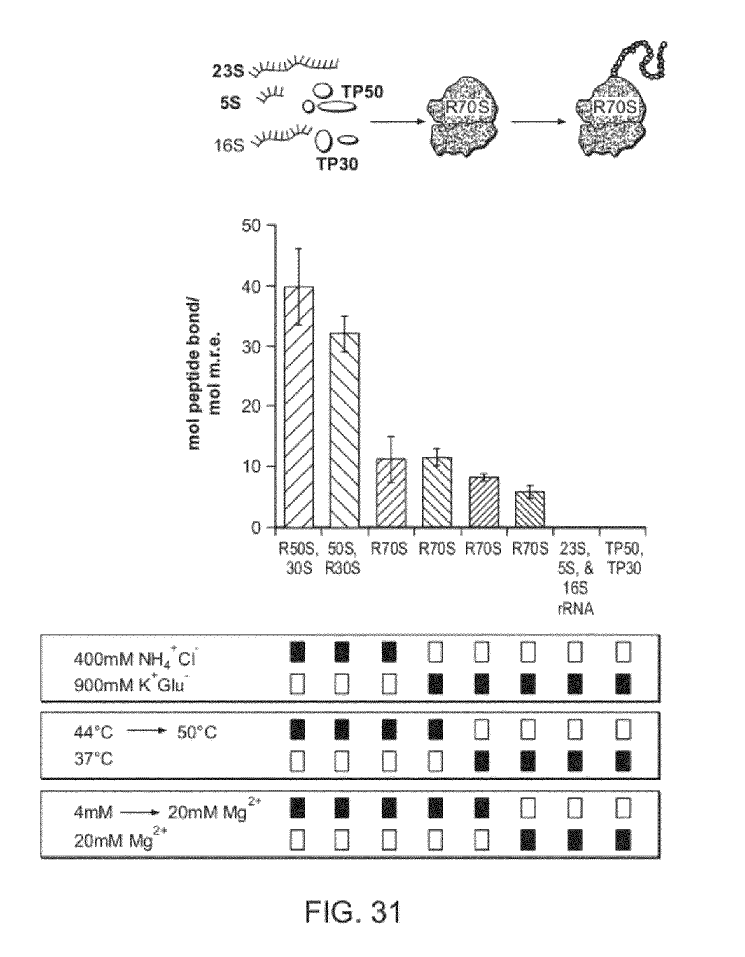

To determine if the more physiological ionic environment could enable one-step 50S subunit assembly, it was tested whether functionally active 50S ribosomal particles could be reconstituted at constant [Mg²⁺] and/or at constant temperature. In all cases of a three-factor factorial design (400 mM NH₄Cl vs. 900 mM KGlu; 44° C.→50° C. vs. 37° C.; and 4 mM→20 mM Mg²⁺ vs. 20 mM Mg²⁺), 50S reconstitution mixtures synthesized protein (FIG. 23). The chemical composition of the protein synthesis system used herein was markedly different than the classical poly(Phe) system (Table 1) and has been shown to provide advantages for protein synthesis and for integrated metabolic activity (Jewett (2004), Supra; Jewett 2008, Supra). Consistent with this hypothesis, Fluc synthesis was not observed for 50S subunits reconstituted in 400 mM NH₄Cl when the chemical composition of the cell-free protein synthesis system described herein was replaced by the ionic environment used in the classical poly(Phe) system (FIG. 29). Another striking observation was that in contrast to reconstitutions carried out at 37° C. with chloride salts, those with glutamate salts had similar protein synthesis activity under two-step and one-step [Mg²⁺] protocols (FIG. 23). The method of making ribosomes described herein is almost 6-fold more active than the classical system when using a one-step incubation at 20 mM Mg²⁺ and 37° C. (FIG. 3). While polyamines and chaperones did not stimulate reconstitution (FIG. 30), the one-step reconstitution method described herein can be used to assemble functionally active 70S ribosomes (FIG. 31) for use in protein synthesis.

TABLE 1

| | [Reagents]_{final} | | [Reagents]_{final} | |
|---|---|---|---|---|
| Reagents | (this work) | | (Nierhaus, 1990)** | |
| Magnesium** | 16.4 | mM | 10.3 | mM |
| Ammonium | 10 | mM | 169 | mM |
| Potassium | 130 | mM | | |
| Glutamate | 154 | mM | | |
| Chloride | | | 170 | mM |
| Acetate** | 2.4 | mM | 9.1 | mM |
| ATP | 1.2 | mM | 1.50 | mM |
| GTP*** | 0.86 | mM | 0.05 | mM |
| UTP*** | 0.86 | mM | | |
| CTP*** | 0.86 | mM | | |
| Folinic acid | 34 | μg/mL | | |
| tRNA | 170.6 | μg/mL | 0.33 | mg/mL |
| Amino acids**** | 2 | mM | 0.08 | μM |
| PEP | 33 | mM | 5.00 | mM |
| Pyruvate Kinase | 1.63 | μg/mL | 0.025 | mg/mL |
| NAD | 0.33 | mM | | |
| CoA | 0.27 | mM | | |
| HEPES-KOH, pH 7.6 (4C) | 57 | mM | 5.83 | mM |
| Tris-HCL, pH 8.0 (4C) | | | 17.08 | mM |
| Tris-OAc, pH 7.5 (4C) | 2.4 | mM | | |
| Oxalic acid | 4 | mM | | |
| Putrescine | 1 | mM | | |
| Spermidine | 1.5 | mM | 0.58 | mM |
| Spermine | | | 0.06 | mM |
| EDTA | | | 0.33 | mM |
| S150 Extract | 0.24 | vol/vol | 0.21 | vol/vol |
| T7 RNA polymerase*** | 0.1 | mg/mL | | |
| Template (DNA or RNA) | 0.013 | mg/mL | 0.83 | mg/mL |

*The ionic milieu was calculated by summing the moles of salt from the energy mix, buffer 3 carryover (which contained the S150 enzyme), and the reconstitution mixture.
**In this work, 2.4 mM Tris-OAc and 2.4 mM magnesium acetate carried over into the protein synthesis reaction from the S150 extract.
***Because this work used a combined transcription and translation system, GTP, UTP, CTP and T7 RNA polymerase were present here, but lacking in the original poly(Phe) system.
****In this work, 20 amino acids were added. In the original poly(Phe) system, only phenylalanine was added.

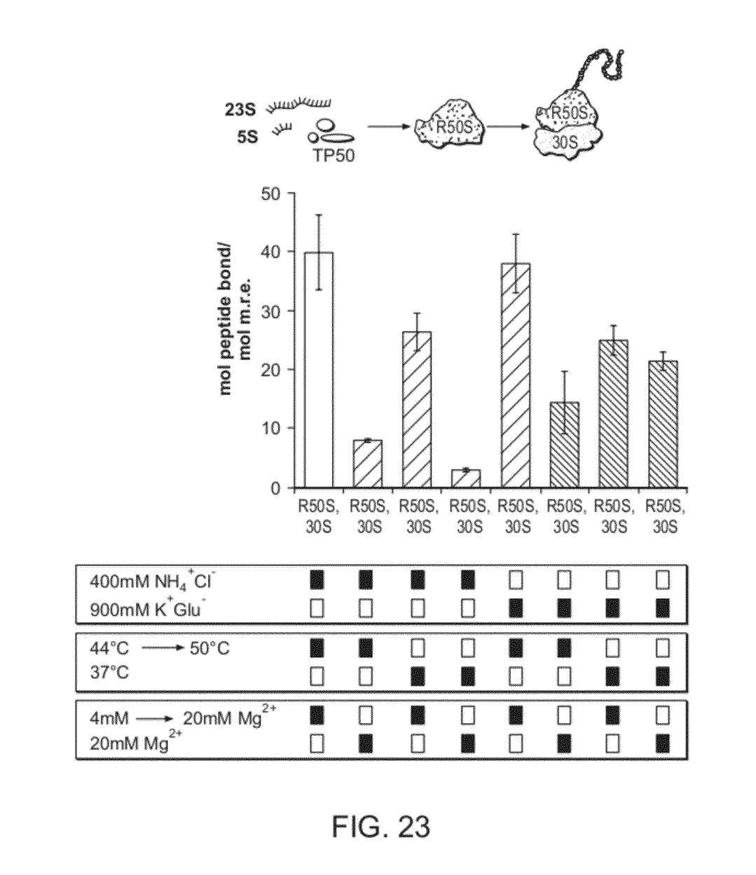

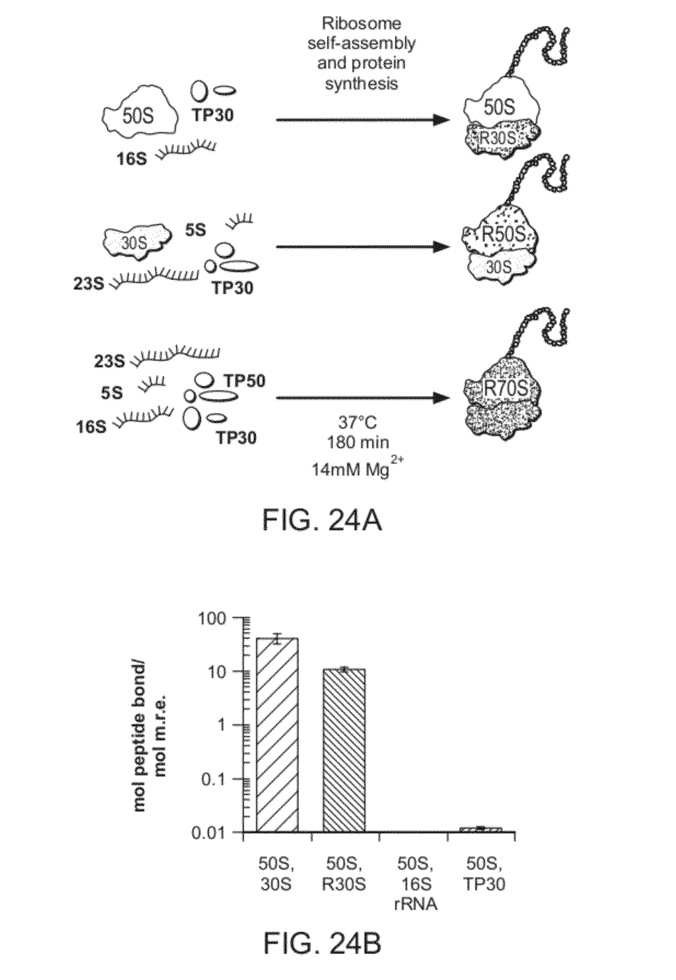

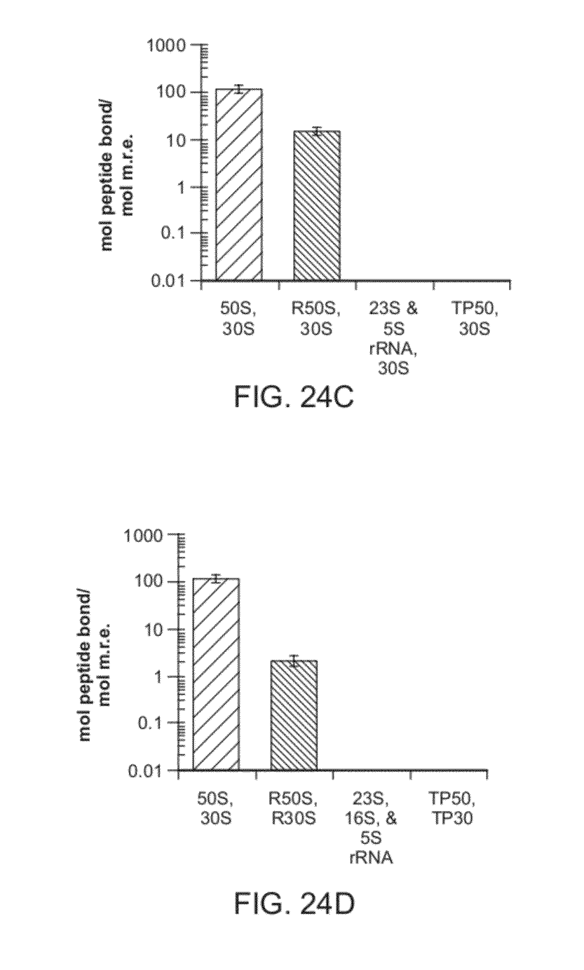

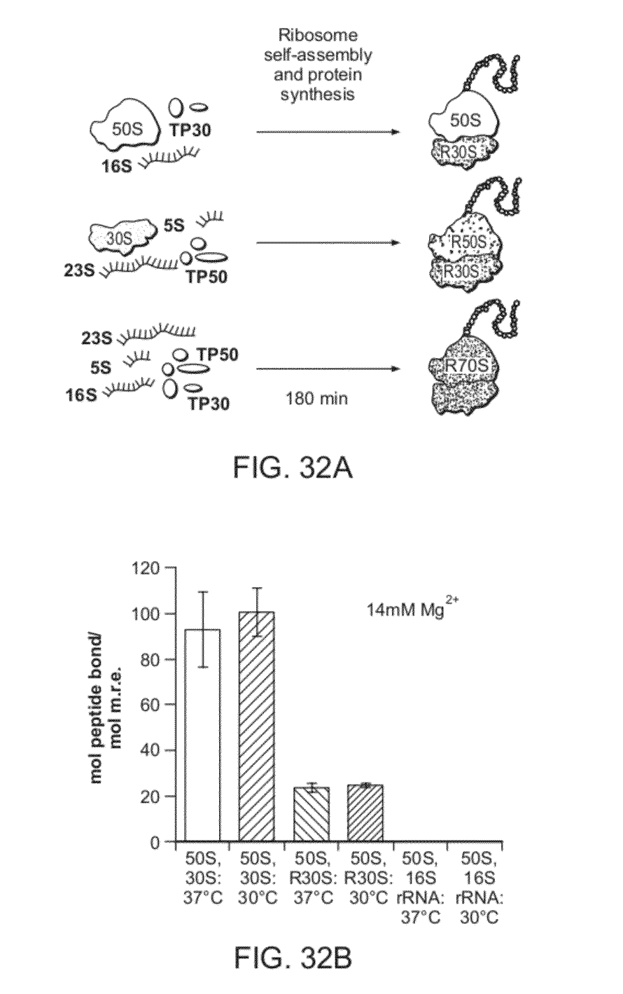

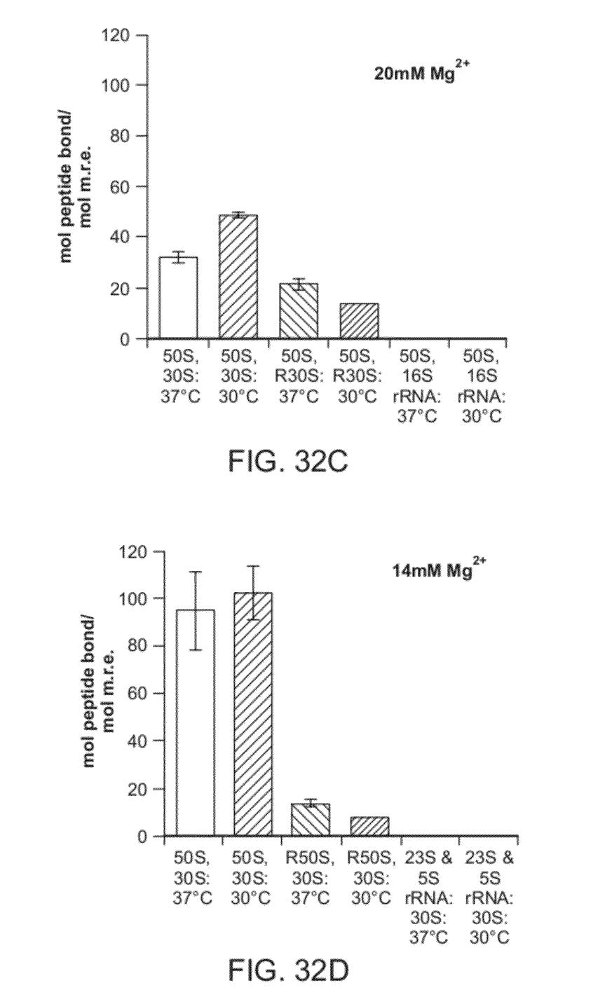

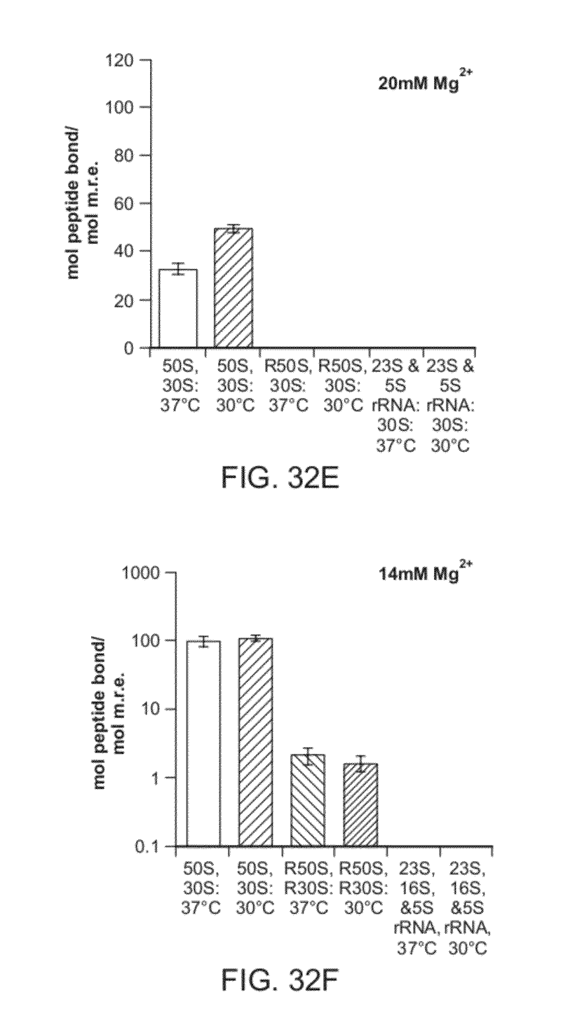

To integrate ribosome self-assembly and protein synthesis, natural rRNAs and total protein mixtures were added directly to the S150 crude extract transcription and translation system (FIG. 24A). Although the optimal [Mg²⁺] and temperature for the one-step ribosome reconstitution protocol (20 mM, 37° C.) and the transcription and translation system (14 mM, 30° C.) were different, protein synthesis activity in a combined self-assembly, transcription, and translation system was observed for 30S and 50S subunits and 70S ribosomes (FIGS. 24B-24D). Higher protein synthesis activities were observed at 37° C. and 14 mM Mg²⁺ (FIG. 32).

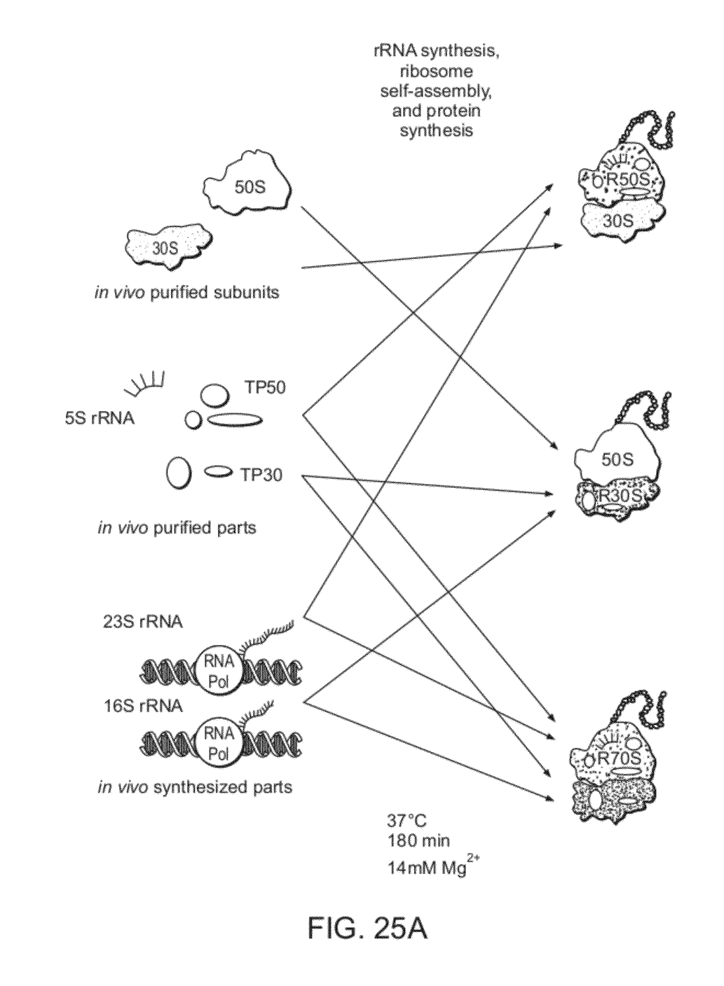

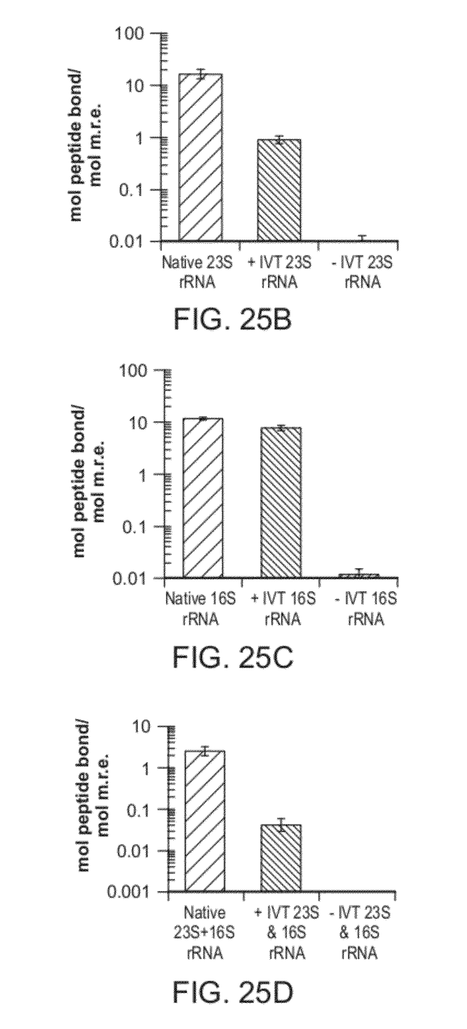

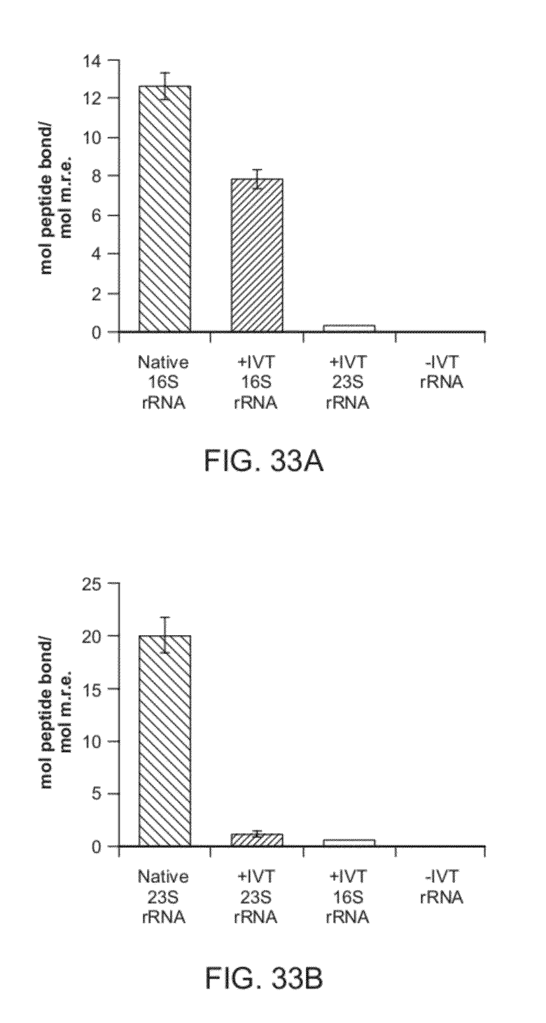

To construct ribosomes, rRNA synthesis, ribosome self-assembly, and protein synthesis were integrated in a single compartment. 23S rRNA synthesis was targeted because it contains the catalytic core of the ribosome and also because reconstitution of functionally active 50S ribosomal particles from in vitro transcribed 23S rRNA is notoriously inefficient (Green, R. & Noller, H. F., In vitro complementation analysis localizes 23S rRNA posttranscriptional modifications that are required for Escherichia coli 50S ribosomal subunit assembly and function. Rna 2, 1011-1021 (1996); Semrad, Supra). Inefficient reconstitution is a result of in vitro transcribed 23S rRNA lacking one to six key nucleoside modifications in a 79 nucleotide region of domain V (Green, Supra). Given the need for nucleoside modification, it was hypothesized that enzymes in the S150 extract could help process, modify, and fold rRNA and/or facilitate assembly of in vitro transcribed 23S rRNA into competent 50S ribosomal particles. Fluc synthesis was investigated in 50S synthesis and self-assembly reactions having native 30S subunits, TP50 and 5S rRNA with or without a plasmid encoding the 23S rRNA gene (FIG. 25A). As a control, reactions without the 23S rRNA gene showed no detectable luciferase synthesis (FIG. 25B). When the 23S rRNA gene was added, 1.7±0.3 mmol Fluc molecules per mol m.r.e. were synthesized (FIG. 25B). As an additional control, it was confirmed that the activity of our in vitro transcribed rRNA was not the result of unmasking some minor level of contaminating native rRNA from the purified TP50 mixture. 50S synthesis and self-assembly reactions carried out with the 16S rRNA gene substituted for the 23S rRNA gene did not synthesize Fluc, confirming that unmasking did not occur (FIG. 33). Supplementation of 100 μM S-adenosyl-methionine, a potential methyl donor for nucleoside modification, did not stimulate the assembly of functional 50S particles. Although PT activity of 50S reconstitution mixtures from classical reconstitutions with in vitro 23S rRNA transcripts was 100-fold diminished relative to those with in vivo derived 23S rRNA (PT activity was 10.000-fold diminished when not stimulated by telithromycin and trimethylamine-oxide) (Green, Supra; Semrad, Supra), the integrated synthesis and assembly approach was only 18-fold diminished in luciferase synthesis (FIG. 25B).

To demonstrate the versatility of the rRNA synthesis and self-assembly approach described herein, the method was used to construct functional 30S ribosomal particles and then 70S ribosomes in a single reaction (FIG. 25A). In 30S subunit synthesis and self-assembly reactions without the 16S rRNA gene, luciferase synthesis was almost undetectable (FIG. 25C). When the 16S rRNA gene was added, Fluc synthesis was approximately 70% the activity observed when natural 16S rRNA was added directly to the system (FIG. 25C). As with 50S subunits, rRNA gene swapping experiments demonstrated no contaminating species (FIG. 33). To build 70S ribosomes, in vitro transcription of 16S and 23S rRNA was combined with self-assembly and protein synthesis (FIG. 25A). Whereas protein synthesis activity was undetectable without rRNA transcription, synthesis of 43±1 mmol peptide bonds/mol m.r.e. was observed when the 23S and 16S rRNA genes were simultaneously transcribed (FIG. 25D). In accordance with other aspects of the invention, rRNA synthesis, ribosome self-assembly, and protein synthesis been co-activated without living cells.

The present invention is directed to an integrated cell-free platform for the synthesis of ribosomal parts and their assembly into functional ribosomes. While focus was given to 16S and 23S rRNA, which comprise the active sites of the ribosome, the general approach could be extended to synthesis of all component parts, particularly since cell-free systems have previously synthesized the 30S subunit proteins (Tian, J. et al., Accurate multiplex gene synthesis from programmable DNA microchips. Nature 432, 1050-1054 (2004)). To help fill gaps in the development of self-replicating systems and mankind's understanding of synthetic life (Forster, A. C. & Church, G. M., Towards synthesis of a minimal cell. Mol Syst Biol 2, 45 (2006); Luisi, P. L., Ferri, F., & Stano, P., Approaches to semi-synthetic minimal cells: a review. Naturwissenschaften 93, 1-13 (2006); Szostak, J. W., Bartel, D. P., & Luisi, P. L., Synthesizing life. Nature 409, 387-390 (2001)), the integrated method described herein could be extended to a ribosome-free ‘PURE’ translation system (Shimizu, Y. et al., Cell-free translation reconstituted with purified components. Nat Biotechnol 19, 751-755 (2001)). However, reconstitution of enzyme activities necessary for active 23S rRNA might be necessary since the PURE system does not utilize a S150 extract. Alternatively, synthetic 23 S rRNAs demonstrating high activity without modifications could be selected by ribosome display (Zahnd, C., Amstutz, P., & Pluckthun, A., Ribosome display: selecting and evolving proteins in vitro that specifically bind to a target. Nat Methods 4, 269-279 (2007)). Bacillus stearothermophilus (Green, R. & Noller, H. F., Reconstitution of functional 50S ribosomes from in vitro transcripts of Bacillus stearothermophilus 23S rRNA. Biochemistry 38, 1772-1779 (1999)) and Thermus aquaticus (Khaitovich, P., Tenson, T., Kloss, P., & Mankin, A. S., Reconstitution of functionally active Thermus aquaticus large ribosomal subunits with in vitro-transcribed rRNA. Biochemistry 38, 1780-1788 (1999)) have quite active ribosomes reconstituted from in vitro transcribed 23S rRNA lacking modifications and could be a good starting point for such directed evolution experiments.

Synthesis of approximately 7000 peptide bonds per active ribosome is the break-even milestone for ribosomes making ribosomes (Table 2). Aspects of the present invention are directed to improving in the rate and yield of protein synthesis and percentage of active ribosomes. Steps include optimization of the ionic environment, synchronizing the rRNA synthesis rate with assembly rates (e.g. by using mutant T7 RNA polymerases (Makarova, O. V., Makarov, E. M., Sousa, R., & Dreyfus, M., Transcribing of Escherichia coli genes with mutant T7 RNA polymerases: stability of lacZ mRNA inversely correlates with polymerase speed. Proc Natl Acad Sci USA 92, 12250-12254 (1995)) or E. coli RNA polymerase), mimicking in vivo enzyme concentrations, identifying factors that stimulate synthesis and self-assembly, stabilizing reaction substrates (Swartz, J., Developing cell-free biology for industrial applications. J Ind Microbiol Biotechnol 33, 476-485 (2006); Jewett (2008), Supra; Noireaux, V. & Libchaber, A., A vesicle bioreactor as a step toward an artificial cell assembly. Proc Natl Acad Sci USA 101, 17669-17674 (2004)), synthesizing the proteins of the synthetic replicon rather than complex eukaryotic reporter proteins, and evolving the ribosome to work better in vitro.

TABLE 2
Total peptide bonds to make all ribosomal proteins = 7128.

| | | | Base | Peptide |
|---|---|---|---|---|
| Function | Products | 54 Genes | Pairs | Bonds |
| Ribosomal Protein Large subunit | RL1 | rplA | 705 | 234 |
| Ribosomal Protein Large subunit | RL2 | rplB | 822 | 273 |
| Ribosomal Protein Large subunit | RL3 | rplC | 630 | 209 |
| Ribosomal Protein Large subunit | RL4 | rplD | 606 | 201 |
| Ribosomal Protein Large subunit | RL5 | rplE | 540 | 179 |
| Ribosomal Protein Large subunit | RL6 | rplF | 534 | 177 |
| Ribosomal Protein Large subunit | RL7 | rplL | 366 | 121 |
| Ribosomal Protein Large subunit | RL9 | rplI | 450 | 149 |
| Ribosomal Protein Large subunit | RL10 | rplJ | 498 | 165 |
| Ribosomal Protein Large subunit | RL11 | rplK | 429 | 142 |
| Ribosomal Protein Large subunit | RL13 | rplM | 429 | 142 |
| Ribosomal Protein Large subunit | RL14 | rplN | 372 | 123 |
| Ribosomal Protein Large subunit | RL15 | rplO | 435 | 144 |
| Ribosomal Protein Large subunit | RL16 | rplP | 411 | 136 |
| Ribosomal Protein Large subunit | RL17 | rplQ | 384 | 127 |
| Ribosomal Protein Large subunit | RL18 | rplR | 354 | 117 |
| Ribosomal Protein Large subunit | RL19 | rplS | 348 | 115 |
| Ribosomal Protein Large subunit | RL20 | rplT | 357 | 118 |
| Ribosomal Protein Large subunit | RL21 | rplU | 312 | 103 |
| Ribosomal Protein Large subunit | RL22 | rplV | 333 | 110 |
| Ribosomal Protein Large subunit | RL23 | rplW | 303 | 100 |
| Ribosomal Protein Large subunit | RL24 | rplX | 315 | 104 |
| Ribosomal Protein Large subunit | RL25 | rplY | 285 | 94 |
| Ribosomal Protein Large subunit | RL27 | rpmA | 258 | 85 |
| Ribosomal Protein Large subunit | RL28 | rpmB | 237 | 78 |
| Ribosomal Protein Large subunit | RL29 | rpmC | 192 | 63 |
| Ribosomal Protein Large subunit | RL30 | rpmD | 180 | 59 |
| Ribosomal Protein Large subunit | RL31 | rpmE | 213 | 70 |
| Ribosomal Protein Large subunit | RL32 | rpmF | 174 | 57 |
| Ribosomal Protein Large subunit | RL33 | rpmG | 168 | 55 |
| Ribosomal Protein Large subunit | RL34 | rpmH | 141 | 46 |
| Ribosomal Protein Large subunit | RL35 | rpmI | 198 | 65 |
| Ribosomal Protein Large subunit | RL36 | rpmJ | 117 | 38 |
| Ribosomal Protein Small subunit | RS1 | rpsA | 1674 | 557 |
| Ribosomal Protein Small subunit | RS2 | rpsB | 726 | 241 |
| Ribosomal Protein Small subunit | RS3 | rpsC | 702 | 233 |
| Ribosomal Protein Small subunit | RS4 | rpsD | 621 | 206 |
| Ribosomal Protein Small subunit | RS5 | rpsE | 504 | 167 |
| Ribosomal Protein Small subunit | RS6 | rpsF | 396 | 131 |
| Ribosomal Protein Small subunit | RS7 | rpsG | 540 | 179 |
| Ribosomal Protein Small subunit | RS8 | rpsH | 393 | 130 |
| Ribosomal Protein Small subunit | RS9 | rpsI | 393 | 130 |
| Ribosomal Protein Small subunit | RS10 | rpsJ | 312 | 103 |
| Ribosomal Protein Small subunit | RS11 | rpsK | 390 | 129 |
| Ribosomal Protein Small subunit | RS12 | rpsL | 375 | 124 |
| Ribosomal Protein Small subunit | RS13 | rpsM | 357 | 118 |
| Ribosomal Protein Small subunit | RS14 | rpsN | 306 | 101 |
| Ribosomal Protein Small subunit | RS15 | rpsO | 270 | 89 |
| Ribosomal Protein Small subunit | RS16 | rpsP | 249 | 82 |
| Ribosomal Protein Small subunit | RS17 | rpsQ | 255 | 84 |
| Ribosomal Protein Small subunit | RS18 | rpsR | 228 | 75 |
| Ribosomal Protein Small subunit | RS19 | rpsS | 279 | 92 |
| Ribosomal Protein Small subunit | RS20 | rpsT | 264 | 87 |
| Ribosomal Protein Small subunit | RS21 | rpsU | 216 | 71 |

The integrated method for rRNA synthesis and self-assembly of functional ribosomes described herein is useful for characterizing ribosomal mutants and to select ribosomes that have enhanced functions or altered chemical properties (Cochella, L. & Green, R., Isolation of antibiotic resistance mutations in the rRNA by using an in vitro selection system. Proc Natl Acad Sci USA 101, 3786-3791 (2004); Wang, K., Neumann, H., Peak-Chew, S. Y., & Chin, J. W., Evolved orthogonal ribosomes enhance the efficiency of synthetic genetic code expansion. Nat Biotechnol 25, 770-777 (2007); Sunguroff, A., Methods of Making Nanotechnological And Macromolecular Biomimetic Structures. WO/2005/123766 (2005); An, W. & Chin, J. W., Synthesis of orthogonal transcription-translation networks. Proc Natl Acad Sci USA 106, 8477-8482 (2009)). For example, a variation on ribosome display is used to evolve ribosomes that are more active under non-physiological pH, temperature, and redox level or to develop a D-amino acid that is tolerant to peptidyl-transferase is engineered for the production of mirror-image polypeptides that have commercial relevance as peptidomimetic drugs, as novel chiral catalysts, in enzyme resistance, and for racemic crystallography (Mandal, K. et al., Racemic crystallography of synthetic protein enantiomers used to determine the X-ray structure of plectasin by direct methods. Protein Sci 18, 1146-1154 (2009)). Relative to in vivo approaches (Dedkova, L. M., Fahmi, N. E., Golovine, S. Y., & Hecht, S. M., Construction of modified ribosomes for incorporation of D-amino acids into proteins. Biochemistry 45, 15541-15551 (2006)), the in vitro method described herein can access greater library diversity (1012 vs. 107), enable fine-tuning of selective pressure, is not limited by dominant lethality or to mutations only in the 16S rRNA (Hui, A. & de Boer, H. A., Specialized ribosome system: preferential translation of a single mRNA species by a subpopulation of mutated ribosomes in Escherichia coli. Proc Natl Acad Sci US A 84, 4762-4766 (1987)), and allows for evolution of all ribosomal parts. According to aspects of the present invention, the methods described herein are directed to the manufacture of synthetic biomolecular machines making natural and non-natural biopolymers and further to methods of making synthetic self-replicating systems.

### REFERENCES

Monro, R. E. & Marcker, K. A., Ribosome-catalysed reaction of puromycin with a formylmethionine-containing oligonucleotide. J Mol Biol 25, 347-350 (1967)
Kim, D. F. & Green, R., Base-pairing between 23S rRNA and tRNA in the ribosomal A site. Mol Cell 4, 859-864 (1999)
Nierhaus, K. H., Reconstitution of ribosomes, in Ribosomes and Protein Synthesis, A Practical Approach. (Oxford University Press, Oxford, 1990).
Maki, J. A., Southworth, D. R., & Culver, G. M., Demonstration of the role of the DnaK chaperone system in assembly of 30S ribosomal subunits using a purified in vitro system. Rna 9, 1418-1421 (2003).
Jelenc, P. C. & Kurland, C. G., Nucleoside triphosphate regeneration decreases the frequency of translation errors. Proc Natl Acad Sci USA 76, 3174-3178 (1979).
Pavlov, M. Y. & Ehrenberg, M., Rate of translation of natural mRNAs in an optimized in vitro system. Arch Biochem Biophys 328, 9-16 (1996).
Shimizu, Y. et al., Cell-free translation reconstituted with purified components. Nat Biotechnol 19, 751-755 (2001).
Shimizu, Y., Kanamori, T., & Ueda, T., Protein synthesis by pure translation systems. Methods 36, 299-304 (2005).
Dawson, R. M. C., Elliott, D. C., Elliott, W. H. and Jones K. M., Data for biochemical research. (Oxford University Press, New York, N.Y., 1990).
Gold, H. A. & Altman, S., Reconstitution of RNAase P activity using inactive subunits from E. coli and HeLa cells. Cell 44, 243-249 (1986).
Green, R. & Noller, H. F., In vitro complementation analysis localizes 23S rRNA posttranscriptional modifications that are required for Escherichia coli 50S ribosomal subunit assembly and function. Rna 2, 1011-1021 (1996).
Semrad, K. & Green, R., Osmolytes stimulate the reconstitution of functional 50S ribosomes from in vitro transcripts of Escherichia coli 23S rRNA. Rna 8, 401-411 (2002).
Rodnina, M. V. & Wintermeyer, W., GTP consumption of elongation factor Tu during translation of heteropolymeric mRNAs. Proc Natl Acad Sci USA 92, 1945-1949 (1995).
Forster, A. C. & Church, G. M., Towards synthesis of a minimal cell. Mol Syst Biol 2, 45 (2006).

### Example V

### Methods

### Isolation of Tightly-Coupled 70S Ribosomes and Ribosomal Subunits

Tight-coupled 70S ribosomes were derived from Escherichia coli strain MRE600 as reported by Blanchard et al. (Supra). Key modifications from the protocols originally described by Powers and Noller (Powers, T. & Noller, H. F., Dominant lethal mutations in a conserved loop in 16S rRNA. Proc Natl Acad Sci USA 87, 1042-1046 (1990)) and Robertson and Wintermeyer (Robertson, J. M. & Wintermeyer, W., Effect of translocation on topology and conformation of anticodon and D loops of tRNAPhe. J Mol Biol 151, 57-79 (1981)) included: (i) cells were lysed by only one pass through a French press at 20,000 p.s.i.g., and (ii) acetate salts were used in place of chloride salts in the sucrose gradients and final resuspension (Acetate Buffer: Tris OAc pH=7.5 @ 4° C., 60 mM NH₄Cl, 7.5 mM Mg(OAc)₂, 0.5 mM EDTA, and 2 mM dithiothreitol). 30S and 50S subunits were derived from ribosomes dialyzed against Acetate Buffer with only 1 mM Mg(OAc)₂ followed by 3 rounds of sucrose density ultracentrifugation in the same buffer. Results from three independent ribosome preparations and subsequent rRNA and total protein preparations were used and averaged to generate the final results shown in the manuscript.

### S150 Crude Extract Preparation

S150 extract was prepared essentially as described (Nierhaus, Supra). The supernatant from the tightly-coupled 70S ribosome pellet, above, was centrifuged at 150,000 g for three hours. The top two-thirds of this supernatant was collected, concentrated 3-fold using Sartorious Vivaspin20 concentration modules (3000 MWCO), and dialyzed overnight against buffer containing 10 mM Tris-OAc pH=7.5 @ 4° C. and 10 mM Mg(OAc)₂ for use as S150 extract.

Isolation of Native rRNA and R-Proteins

Total proteins of the 50S subunit, total proteins of the 30S subunit, natural 23S rRNA, natural 16S rRNA, and natural 5S rRNA were prepared as described by Nierhaus (Supra).

### Ribosome Reconstitution

Briefly, the 50S reconstitution was a two-step procedure. Id. In a total volume of 15 μL, 0.5 A₂₆₀ Units of 23S rRNA, 0.02 A₂₆₀ Units of 5S rRNA were incubated with 1.2 equivalents of TP50 in 20 mM Tris-OAc (pH 7.4 at 37° C.), 4 mM Mg(OAc)₂, 400 mM NH₄Cl, 0.2 mM EDTA, 5 mM 2-mercaptoethanol at 44° C. for 20 minutes. Then, the Mg(OAc)₂ concentration was raised to 20 mM and the temperature was raised to 50° C. for 90 minutes. The classical 30S reconstitution was a one-step procedure. Id. In a total volume of 15 μL, 0.5 A₂₆₀ Units of 16S rRNA were incubated with 1.2 equivalents of TP30 in 20 mM Tris-OAc (pH 7.4 at 37° C.), 20 mM Mg(OAc)₂, 400 mM NH₄Cl, 0.2 mM EDTA, 5 mM 2-mercaptoethanol at 40° C. for 20 minutes. Following reconstitution, subunit concentrations were calculated from A₂₆₀ measurements (1 A₂₆₀ Unit of 50S=36 pmol, 1 A₂₆₀ Unit of 30S=72 pmol).

Variations on the classical 50S reconstitution method included: (i) substituting NH₄OAc for NH₄Cl, (ii) substituting potassium glutamate for NH₄Cl, (iii) supplementing the reaction with 1.5 mM spermidine and 1 mM putrescine, (iv) supplementing the reaction with chaperones (1.5 pmol DnaK, 1.5 pmol DnaJ, 3 pmol GrpE, 1.5 pmol GroEL, and 1.5 pmol GroES were added per 0.75 pmol 23S rRNA, along with 1 mM ATP), (v) using a one-step 37° C. incubation, and (vi) using a one-step constant Mg(OAc)₂ concentration incubation.

### Cell-Free Protein Synthesis

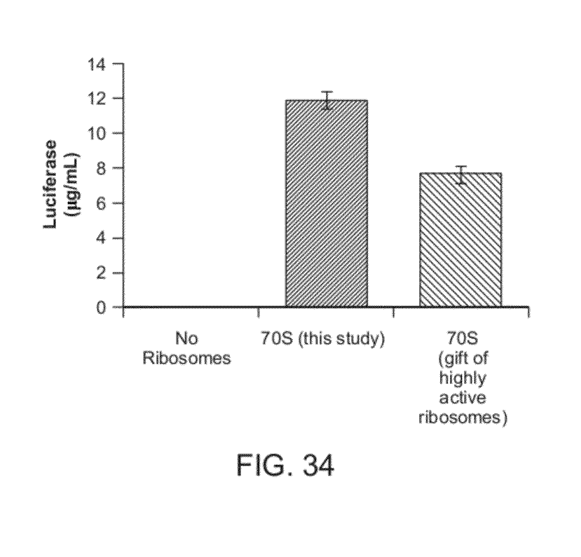

Following assembly, reconstitution mixtures were added directly to an S150-based cell-free transcription and translation system designed to mimic the cytoplasm (See full Methods). S150 extract, lacking ribosomes, was prepared from E. coli strain MRE600. Id. Protein synthesis was carried out for 2 hours at 30° C. and firefly luciferase activity was quantified using the ONE-Glo™ Luciferase Assay System (Promega, Madison, Wis.). Control transcription and translation reactions without ribosomes or without Fluc template DNA had immeasurable luminescence. In the case of 50S or 30S assembly, background protein production levels with 30S or 50S subunits only, respectively, were subtracted from measured values. Comparison of luciferase production from an independent set of highly active ribosomes was used to confirm ribosome quality (FIG. 34). Subunit molar ratios for the various reconstitution experiments were 50S:R30S=2:1, R50S:30S=1:4, and R50S:R30S=1:2

Combined transcription and translation reactions were carried out in 1.5 ml Eppendorf tubes (Jewett (2004), Supra). The standard reaction mixture contained the following components: 1.2 mM ATP, 0.85 mM each of GTP, UTP and CTP, 34 μg/ml folinic acid, 170.6 μg/ml E. coli tRNA mixture (Roche, Indianapolis, Ind.), 16.6 μg/ml plasmid DNA (pk7LUC or pk7GFP, see below), 100 μg/m1 T7 RNA polymerase, 2 mM each of 20 unlabeled amino acids, 33 mM phosphoenolpyruvate (Roche, Indianapolis, Ind.), 0.33 mM NAD, 0.26 mM CoA, 130 mM potassium glutamate, 10 mM ammonium glutamate, 14 mM magnesium glutamate, 1.5 mM spermidine, 1 mM putrescine, 4 mM sodium oxalate, and 0.24 volume of S150 extract. Reaction volumes were 15 μl. Except where specified, reagents were from Sigma (St. Louis, Mo.).

Plasmid pk7LUC (encoding the gene for Fluc) and pk7GFP (encoding the gene for eGFP) were constructed for this study. The Fluc gene (from pZE21-luc) (Lutz, R. & Bujard, H., Independent and tight regulation of transcriptional units in Escherichia coli via the LacR/O, the TetR/O and AraC/I1-I2 regulatory elements. Nucleic Acids Res 25, 1203-1210 (1997)) and the eGFP gene (from pZE21G) (Isaacs, F. J. et al., Engineered riboregulators enable post-transcriptional control of gene expression. Nat Biotechnol 22, 841-847 (2004)) were amplified by PCR and sub-cloned into the expression plasmid pK7 after removing the CAT-encoding sequence from pk7CAT (Jewett (2008), Supra) to yield pk7LUC and pk7GFP. The following primer sequences were used: 5′ primer for Fluc, 5′-GGT-GGT-CAT-ATG-GAA-GAC-GCC-AAA-AAC-AT-3′ (SEQ ID NO:1); 3′ primer for Fluc, 5′-GGT-GGT-GTC-GAC-TTA-CAA-TTT-GGA-CTT-TCC-GC-3′ (SEQ ID NO:2); 5′ primer for GFP, 5′-GGT-GGT-TCT-AGA-AAT-AAT-TTT-GTT-TAA-CTT-TAA-GAA-GGA-GAT-ATA-CAT-ATG-CGT-AAA-GGA-GAA-GAA-CTT-T-3′ (SEQ ID NO:3); 3′ primer for GFP, 5′-GGT-GGT-GTC-GAC-TTA-TTT-GTA-TAG-TTC-ATC-CAT-GCC-A-3′ (SEQ ID NO:4). Nucleotide sequences of recombinant genes were verified by DNA sequencing (Agencourt Bioscience). T7 RNA polymerase was prepared as described earlier (Jewett (2004), Supra).

### Reporter Protein Quantification

Bioluminescence was measured using the SpectraMax M5 plate reader (Molecular Devices, Sunnyvale, Calif.). Forty microliters of ONE-Glo™ Luciferase Assay reagent were added to the entire 15 μL cell-free reaction and luminescence was measured immediately thereafter. Relative Light Units were converted to an estimate of protein concentration using a standard curve created from dilutions of purified QuantiLum® Recombinant Fluc (Promega, Madison, Wis.) (1RLU is approximately 15 fg).

The active amount of eGFP synthesized in each cell-free protein synthesis reaction was calculated by measuring the fluorescence after a maturation period of 8 hours at 4° C. Longer maturation times did not increase fluorescence. By calculating the signal to background ratio (S-B)/B for multiple wavelength pairs, the optimum excitation and emission spectra were determined to be 490 nm/520 nm. Fluorescence was measured using the SpectraMax M5 plate reader (Molecular Devices, Sunnyvale, Calif.). Fluorescence was converted to an estimate of protein concentration using a standard curve created from dilutions of purified recombinant GFP (Roche Diagnostics Corporation, Indianapolis, Ill.) (Excitation/Emission=395 nm/510 nm).

### Combined Ribosome Self-Assembly and Protein Synthesis

To combine 50S subunit self-assembly and protein synthesis, natural 23S rRNA, natural 5S rRNA, TP50, and 30S subunits were added directly to S150 extract on ice. Next, a pre-chilled reagent mixture at 4° C. comprising the necessary salts, energy substrates, T7 RNA polymerase, and pk7LUC was added. The reaction was mixed to homogeneity by pipetting and then incubated for 3 hours at 37° C. Similarly, 30S subunit and 70S ribosome assembly and protein synthesis activity were tested. Using different total amounts of reconstituted ribosomes (e.g., R50S:30S=0.75 pmol:3 pmol or 4.5 pmol:9 pmol) resulted in the same number of peptide bonds synthesized per maximum ribosome equivalent. Increasing the molar ratio of self-assembled to native ribosomes from 1:2 to 1:4 did not increase protein synthesis activity (e.g. moles peptide bonds per mole m.r.e. was the same for R50S:30S=1:2=1:4).

rRNA Synthesis, Ribosome Self-Assembly, and Protein Synthesis

Combined self-assembly and protein synthesis reactions were set-up as described above. However, the in vivo derived rRNA to be synthesized was not added. In this case, a plasmid encoding the target rRNA gene behind the T7 promoter was added (pCW1 (Weitzmann, C. J., Cunningham, P. R., & Ofengand, J., Cloning, in vitro transcription, and biological activity of Escherichia coli 23S ribosomal RNA. Nucleic Acids Res 18, 3515-3520 (1990)) for full length 23S rRNA and pWK1 (Krzyzosiak, W. et al., In vitro synthesis of 16S ribosomal RNA containing single base changes and assembly into a functional 30S ribosome. Biochemistry 26, 2353-2364 (1987)) for full length 16S rRNA). The final concentration of pCW1 was 16.6 μg/mL and pWK1 was 13.3 μg/ml. Competent ribosomes that self-assembled from in vitro transcribed rRNA engaged on Fluc mRNA to synthesize active firefly Fluc. Linearized plasmids of pCW1 (digested with Afl II) and pWK1 (digested with BSU361) were 80±6% and 60±7%, respectively, less active in Fluc synthesis relative to adding the entire plasmid. Without intending to be bound by scientific theory, this may be a result of nucleases in the extract and indicates that rRNA processing enzymes in the S150 extract were present and active. Subunit molar ratios: 50S:R30S=2:1, R50S:30S=1:2, and R70S(R50S:R30S=1:2).

What is claimed is:

1. A method of making a pharmaceutical compound comprising:
providing ribosomal proteins that assemble to form one or more ribosomal subunits;
transcribing synthetic rRNA in the presence of the ribosomal proteins to form the ribosomal subunits;
allowing the ribosome to self-assemble;
providing a nucleic acid sequence encoding a pharmaceutical compound; and
allowing the ribosome to translate the nucleic acid sequence to produce the pharmaceutical compound.

2. The method of claim 1, wherein the ribosomal proteins are synthetic.

3. The method of claim 1, wherein the pharmaceutical compound is an non-natural biopolymer.

4. The method of claim 1, wherein the pharmaceutical compound is a natural biopolymer.

5. The method of claim 1, wherein the pharmaceutical compound contains one or more D-amino acids.

6. A method of making an in vitro assembled ribosomal subunit comprising:
providing polypeptides that assemble to form a ribosomal subunit;
contacting the polypeptides that assemble to form the ribosomal subunit with ribosomal RNAs (rRNAs);
incubating the polypeptides and rRNAs at a constant temperature and a constant Mg²⁺ concentration; and
allowing assembly of the ribosomal subunit.

7. The method of claim 6, wherein the incubating is performed at about 37° C.

8. The method of claim 6, wherein the Mg²⁺ concentration is about 20 mM.

9. The method of claim 6, wherein the ribosomal subunit is a 50S subunit or a 30S subunit.

10. The method of claim 6, wherein the rRNAs are natural.

11. The method of claim 6, wherein the rRNAs are synthetic.

12. The method of claim 6, wherein both 30S and 50S subunits are assembled.

13. The method of claim 6, wherein one or more of the polypeptides that assemble to form the ribosomal subunits are synthetic.

14. A method of making an in vitro assembled ribosome comprising:
providing polypeptides that assemble to form one or more ribosomal subunits;
transcribing synthetic rRNA in the presence of the polypeptides that assemble to form the ribosomal subunits; and
allowing the ribosome to self-assemble.

15. The method of claim 14, wherein the synthetic rRNA is selected from the group consisting of 16S rRNA, 23S rRNA, 5S rRNA or any combination thereof.

16. The method of claim 14, wherein the polypeptides and rRNA assemble to form one or both of 30S subunit and 50S subunit.

17. A method of in vitro translation comprising:
providing polypeptides that assemble to form ribosomal subunits in a vessel;
providing transcription reagents and a nucleic acid sequence that encodes rRNA in the vessel;
providing tRNA, a polymerase, NTPs, amino acids and a nucleic acid sequence encoding a protein in the vessel;
allowing assembly of ribosomal subunits and rRNA to form a ribosome; and
allowing the ribosome to translate the protein encoded by the nucleic acid sequence.

18. The method of claim 17, wherein the contents of the vessel are incubated at a temperature between about 30° C. and 37° C.

19. The method of claim 17, wherein the contents of the vessel are incubated in the presence of Mg²⁺ present at a concentration between about 10 mM and 25 mM.

20. The method of claim 17, wherein the contents of the vessel are incubated at a temperature of about 37° C. in the presence of about 14 mM Mg²⁺.

21. The method of claim 17, wherein the at least some natural rRNAs are provided in the vessel.

22. The method of claim 17, wherein all the rRNAs are transcribed in the vessel.

23. The method of claim 17, wherein a sequence-defined non-natural or natural biopolymer is synthesized.

24. The method of claim 17, wherein the protein has one or more biological activities.

25. The method of claim 24, wherein the one or more biological activities includes a pharmaceutical activity.

26. A method of making an in vitro assembled ribosome comprising:
translating ribosomal proteins that assemble to form one or more ribosomal subunits;
transcribing synthetic rRNA in the presence of the ribosomal proteins to form the ribosomal subunits; and
allowing the ribosome to self-assemble.

27. The method of claim 26, wherein the synthetic rRNA is selected from the group consisting of 16S rRNA, 23S rRNA, 5S rRNA or any combination thereof.

28. The method of claim 26, wherein the ribosomal proteins include any one or more of known ribosomal proteins.

29. The method of claim 26, wherein the ribosomal proteins include all known ribosomal proteins.

30. The method of claim 26, wherein the ribosomal proteins and the rRNA assemble to form one or both of 30S subunit and 50S subunit.

31. A method of making an in vitro assembled ribosome comprising:
translating the ribosomal proteins in the presence of rRNA that assemble to form one or more ribosomal subunits;
providing rRNA that assembles to form the ribosomal subunits; and
allowing the ribosome to self-assemble.

32. The method of claim 31, wherein the synthetic ribosomal proteins include any one or more of known ribosomal proteins.

33. The method of claim 31, wherein the synthetic ribosomal proteins include all known ribosomal proteins.

34. The method of claim 31, wherein the components assemble to form one or both of 30S subunit and 50S subunit.

35. A method of making a non-natural biopolymer comprising:
providing ribosomal proteins that assemble to form one or more ribosomal subunits;
transcribing synthetic rRNA in the presence of the ribosomal proteins to form the ribosomal subunits;
allowing the ribosome to self-assemble;
providing a nucleic acid sequence encoding a pharmaceutical compound; and
allowing the ribosome to translate the nucleic acid sequence to produce the non-natural biopolymer.
